SEQUENCE LISTING (68 sequences)

SEQ ID NO: 1 - protein, 199 aa, Archaeoglobus fulgidus
MERRTLIMEVLFEAKVGDITLKLAQGDITQYPAKAIVNAANKRLEHGGGVAYAIAKACAGDAGLYTEISKKAMREQFGRDYIDHGEVVVTPAMNLEERGIKYVFHTVGPICSGMWSEELKEKLYKAFLGPLEKAEEMGVESIAFPAVSAGIYGCDLEKVVETFLEAVKNFKGSAVKEVALVIYDRKSAEVALKVFERSL

SEQ ID NO: 2 - protein, 84 aa, Homo sapiens
MGNGEYAWYYEGRNGWWQYDERTSRELEDAFSKGKKNTEMLIAGFLYVADLENMVQYRRNEHGRRRKIKRDIIDIPKKGVAGLR

SEQ ID NO: 3 - protein, 60 aa, Homo sapiens
MDSVLQGSEGNKVKRTSCMYGANCYRKNPVHFQHFSHPGDSDYGGVQIVGQDETDDRPEC

SEQ ID NO: 4 - DNA, 14358 bp, Artificial Sequence
  synthetic construct
gatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggcccgggttaattaaggaaagggctagatcattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagcaagctcatggctgactaattttttttatttatgcagaggccgaggccgcctcggcctctgagctattccagaagtagtgaggaggcttttttggaggcctaggcttttgcaaaaagctccccgtggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgacatgattacgaatttcacaaataaagcatttttttcactgcattctagttgtggtttgtccaaactcatcaatgtatcttatcatgtctggatcaactggataactcaagctaaccaaaatcatcccaaacttcccaccccataccctattaccactgccaattacctgtggtttcatttactctaaacctgtgattcctctgaattattttcattttaaagaaattgtatttgttaaatatgtactacaaacttagtagttggaagggctaattcactcccaaagaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgccgaattcacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggactaggatcctttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagctcggtacccgggtcgaggtaggcgtgtacggtgggaggcctatataagcagagctcgtttagtgaaccgtcagatcgcctggagacgccatccacgctgttttgacctccatagaagacaccgggaccgatccagcctccgcggccccgaactagtggccaccatggactacaaggatgacgatgacaagcttgaagcaaccatggaacggcgtactttaatcatggaggtgctttttgaggccaaggttggggacatcactctgaagctcgctcagggggacatcacccaatacccggcaaaggcaattgtcaacgcggccaacaagaggctggagcacggcggaggggtggcttatgccatcgcaaaagcgtgtgcaggagatgccgggctctacacggaaatcagcaaaaaggccatgagagagcagtttggaagagactacatcgaccacggcgaggtcgttgttacaccggccatgaacctcgaggagaggggaataaagtacgttttccacaccgtggggccgatatgcagcggcatgtggagcgaagaactgaaagagaagctttacaaggcttttcttggcccgctggagaaggcggaggagatgggcgtcgaatccatagccttccctgctgtgagcgctgggatatacggctgtgatctggaaaaggttgttgagacgttcctcgaagctgtgaagaacttcaagggttcggctgtcaaggaagtcgcgcttgtaatctacgacagaaagtctgcggaggtggcgctgaaggtctttgagaggagtcttctagaattctcgacagggcatggcaccggcagcaccggcagcggcagctccggtaccgcctcctccgaatcgaccggtagttcaatggcgagcaagagcgaggaggtcatcaaagagttcatgcgcttcaaggtgcgcttggagggctccatgaacggccacgagttcgagatcgagggcgagggcgagggccgcccttacgagggcacccagaccgccaagctgaaggtgaccaagggcggccccctgcccttcgcctgggacatcctgtcccccctgatcatgtacggctccaagatgtacgtgaagcaccccgccgacgtccccgattacatgaagctgtccttccccgagggcttcaagtgggagcgcgtgatgcacttcgaggacggcggtctggtgaccgcgacacaggactcctccctgcaggacggcacgctgatctacaaggtgaagatgcgcggcaccaacttcccccccgacggccccgtaatgcagaagaagaccttgggctgggattatgccaccgagcgcctgtaccccgaagaaggcgtgctgaagggcgagctcctggggcgcctgaagctgaaggacggcggcctcaacctggtggagtccaagaccatctacatggccaagaagcccgtgcaactgcccggctactacttcgtggacaccaagctggacatcacctcccacaacgaggactacaccatcgtggaacagtacgagcgctccgagggccgccaccacctggggcattgaactagaggaattccgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgatgccaccatgtacccatacgatgttccagattacgctcttgaagcaaccggaaatggtgaatatgcatggtattatgaaggaagaaatgggtggtggcagtacgatgagcgcactagtagagagctggaagatgctttttccaaaggtaaaaagaacactgaaatgttaattgctggctttctgtatgtcgctgatcttgaaaacatggttcaatataggagaaatgaacatggacgtcgcaggaagattaagcgagatataatagatataccaaagaagggagtagctggacttaggctagaattctcgacagggcatggcaccggcagcaccggcagcggcagctccggtaccgcctcctccgaatcgaccggtagttcaaccatcaaagagttcatgcgcttcaaggtgcgcatggagggctccatgaacggccacgagttcgagatcgagggcgagggcgagggccgcccctacgagggcacccagaccgccaagctgaaggtgaccaagggcggccccctgccctttgcctgggacatcctgtccccccagatcatgtacggctccaaagcgtacgtgaggcaccccgccgacatccccgattacaagaagctgcccttccccgagggcttcaagtgggagcgcgtgatgaacttcgaagacggcggtctggtgaccgttacccaggactcctccctgcaggacggcacgctgatctgcaaggtgaagattcgcggcaccaacttcccccccgacggccccgtaatgcagaagaagaccatgggctgggaggcctccaccgagatgctgtaccccaaggacggcgtgctgaagggccatagctatcaggccctgaagctgaaggacggcggccactacctggtggagttcgagaccatctacatggccaagaagcccgtgcaactgcccggcgattactgtgtggacaccaagctggacatcacctcccacaacgaggactacaccatcgtggaacagtacgggcgctccgagggccgccaccgtctgggcatggacgagcggtacaagtaagactagaggaattccgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgccaccatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgaactgtacaagcctaaaaagaaacgtaaagttgatagtaatgattaattaaactagaaattctaccgggtaggggaggcgcttttcccaaggcagtctggagtaacccacccaagatctggcctccgcgccgggttttggcgcctcccgcgggcgcccccctcctcacggcgagcgctgccacgtcagacgaagggcgcagcgagcgtcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgattatataaggacgcgccgggtgtggcacagctagttccgtcgcagccgggatttgggtcgcggttcttgtttgtggatcgctgtgatcgtcacttggtgagtagcgggctgctgggctggccggggctttcgtggccgccgggccgctcggtgggacggaagcgtgtggagagaccgccaagggctgtagtctgggtccgcgagcaaggttgccctgaactgggggttggggggagcgcagcaaaatggcggctgttcccgagtcttgaatggaagacgcttgtgaggcgggctgtgaggtcgttgaaacaaggtggggggcatggtgggcggcaagaacccaaggtcttgaggccttcgctaatgcgggaaagctcttattcgggtgagatgggctggggcaccatctggggaccctgacgtgaagtttgtcactgactggagaactcggtttgtcgtctgttgcgggggcggcagttatggcggtgccgttgggcagtgcacccgtacctttgggagcgcgcgccctcgtcgtgtcgtgacgtcacccgttctgttggcttataatgcagggtggggccacctgccggtaggtgtgcggtaggcttttctccgtcgcaggacgcagggttcgggcctagggtaggctctcctgaatcgacaggcgccggacctctggtgaggggagggataagtgaggcgtcagtttctttggtcggttttatgtacctatcttcttaagtagctgaagctccggttttgaactatgcgctcggggttggcgagtgtgttttgtgaagttttttaggcaccttttgaaatgtaatcatttgggtcaatatgtaattttcagtgttagactagtaaattgtccgctaaattctggccgtttttggcttttttgttagacgaagcttggtaccgagctcggatctccaccccgtaccggtcctgcagtcgaattcaccatgtctagactggacaagagcaaagtcataaacggagctctggaattactcaatggtgtcggtatcgaaggcctgacgacaaggaaactcgctcaaaagctgggagttgagcagcctaccctgtactggcacgtgaagaacaagcgggccctgctcgatgccctgccaatcgagatgctggacaggcatcatacccacttctgccccctggaaggcgagtcatggcaagactttctgcggaacaacgccaagtcataccgctgtgctctcctctcacatcgcgacggggctaaagtgcatctcggcacccgcccaacagagaaacagtacgaaaccctggaaaatcagctcgcgttcctgtgtcagcaaggcttctccctggagaacgcactgtacgctctgtccgccgtgggccactttacactgggctgcgtattggaggaacaggagcatcaagtagcaaaagaggaaagagagacacctaccaccgattctatgcccccacttctgagacaagcaattgagctgttcgaccggcagggagccgaacctgccttccttttcggcctggaactaatcatatgtggcctggagaaacagctaaagtgcgaaagcggcgggccgaccgacgcccttgacgattttgacttagacatgctcccagccgatgcccttgacgactttgaccttgatatgctgcctgctgacgctcttgacgattttgaccttgacatgctccccgggtaactaagtaaggatccgcggccgcactagaggaattccgcccctctccctcccccccccctaacgttactggccgaagccgcttggaataaggccggtgtgtgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtagtcaacaaggggctgaaggatgcccagaaggtaccccattgtatgggaatctgatctggggcctcggtgcacatgctttacatgtgtttagtcgaggttaaaaaaacgtctaggccccccgaaccacggggacgtggttttcctttgaaaaacacgatgataagcttaccggtccaccatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgatgtacaagtaaagcggccgcgactctagatcataatcagccataccacatttgtagaggttttacttgctttaaaaaacctcccacacctccccctgaacctgaaacataaaatgaatgcaattgttgttgtttagtccctcccaattcgatatcaagcttatcgatcgatagatcctaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctgagatcctttaagaccaatgacttacaaggcagctgta

SEQ ID NO: 5 - DNA, 6732 bp, Artificial Sequence
  synthetic construct
atgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggccaggacccaacgctgcccgagatgcgccgcgtgcggctgctggagatggcggacgcgatggatatgttctgccaagggttggtttgcgcattcacagttctccgcaagaattgattggctccaattcttggagtggtgaatccgttagcgaggtgccgccggcttccattcaggtcgaggtggcccggctccatgcaccgcgacgcaacgcggggaggcagacaaggtatagggcggcgcctacaatccatgccaacccgttccatgtgctcgccgaggcggcataaatcgccgtgacgatcagcggtccagtgatcgaagttaggctggtaagagccgcgagcgatccttgaagctgtccctgatggtcgtcatctacctgcctggacagcatggcctgcaacgcgggcatcccgatgccgccggaagcgagaagaatcataatggggaaggccatccagcctcgcgtcgcgaacgccagcaagacgtagcccagcgcgtcggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatctcgatcccgcgaaattaatacgactcactataggggaattgtgagcggataacaattcccctctagaaataattttgtttaactttaagaaggagatataccatgggccatcatcatcatcatcatcatcatcatcacagcagcggccatatcgacgacgacgacaagcatatgctcgagggaaatggtgaatatgcatggtattatgaaggaagaaatgggtggtggcagtacgatgagcgcactagtagagagctggaagatgctttttccaaaggtaaaaagaacactgaaatgttaattgctggctttctgtatgtcgctgatcttgaaaacatggttcaatataggagaaatgaacatggacgtcgcaggaagattaagcgagatataatagatataccaaagaagggagtagctggacttaggctagaattctcgacagggcatggcaccggcagcaccggcagcggcagctccggtaccgcctcctccgaatcgaccggtagttcaatggcgagcaagagcgaggaggtcatcaaagagttcatgcgcttcaaggtgcgcttggagggctccatgaacggccacgagttcgagatcgagggcgagggcgagggccgcccttacgagggcacccagaccgccaagctgaaggtgaccaagggcggccccctgcccttcgcctgggacatcctgtcccccctgatcatgtacggctccaagatgtacgtgaagcaccccgccgacgtccccgattacatgaagctgtccttccccgagggcttcaagtgggagcgcgtgatgcacttcgaggacggcggtctggtgaccgcgacacaggactcctccctgcaggacggcacgctgatctacaaggtgaagatgcgcggcaccaacttcccccccgacggccccgtaatgcagaagaagaccttgggctgggattatgccaccgagcgcctgtaccccgaagaaggcgtgctgaagggcgagctcctggggcgcctgaagctgaaggacggcggcctcaacctggtggagtccaagaccatctacatggccaagaagcccgtgcaactgcccggctactacttcgtggacaccaagctggacatcacctcccacaacgaggactacaccatcgtggaacagtacgagcgctccgagggccgccaccacctggggcattgaggatccggctgctaacaaagcccgaaaggaagctgagttggctgctgccaccgctgagcaataactagcataaccccttggggcctctaaacgggtcttgaggggttttttgctgaaaggaggaactatatccggatatcccgcaagaggcccggcagtaccggcataaccaagcctatgcctacagcatccagggtgacggtgccgaggatgacgatgagcgcattgttagatttcatacacggtgcctgactgcgttagcaatttaactgtgataaactaccgcattaaagcttatcgatgataagctgtcaaacatgagaattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactg

SEQ ID NO: 6 - DNA, 6726 bp, Artificial Sequence
  synthetic construct
atgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggccaggacccaacgctgcccgagatgcgccgcgtgcggctgctggagatggcggacgcgatggatatgttctgccaagggttggtttgcgcattcacagttctccgcaagaattgattggctccaattcttggagtggtgaatccgttagcgaggtgccgccggcttccattcaggtcgaggtggcccggctccatgcaccgcgacgcaacgcggggaggcagacaaggtatagggcggcgcctacaatccatgccaacccgttccatgtgctcgccgaggcggcataaatcgccgtgacgatcagcggtccagtgatcgaagttaggctggtaagagccgcgagcgatccttgaagctgtccctgatggtcgtcatctacctgcctggacagcatggcctgcaacgcgggcatcccgatgccgccggaagcgagaagaatcataatggggaaggccatccagcctcgcgtcgcgaacgccagcaagacgtagcccagcgcgtcggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatctcgatcccgcgaaattaatacgactcactataggggaattgtgagcggataacaattcccctctagaaataattttgtttaactttaagaaggagatataccatgggccatcatcatcatcatcatcatcatcatcacagcagcggccatatcgacgacgacgacaagcatatgctcgagggaaatggtgaatatgcatggtattatgaaggaagaaatgggtggtggcagtacgatgagcgcactagtagagagctggaagatgctttttccaaaggtaaaaagaacactgaaatgttaattgctggctttctgtatgtcgctgatcttgaaaacatggttcaatataggagaaatgaacatggacgtcgcaggaagattaagcgagatataatagatataccaaagaagggagtagctggacttaggctagaattctcgacagggcatggcaccggcagcaccggcagcggcagctccggtaccgcctcctccgaatcgaccggtagttcaaccatcaaagagttcatgcgcttcaaggtgcgcatggagggctccatgaacggccacgagttcgagatcgagggcgagggcgagggccgcccctacgagggcacccagaccgccaagctgaaggtgaccaagggcggccccctgccctttgcctgggacatcctgtccccccagatcatgtacggctccaaagcgtacgtgaggcaccccgccgacatccccgattacaagaagctgcccttccccgagggcttcaagtgggagcgcgtgatgaacttcgaagacggcggtctggtgaccgttacccaggactcctccctgcaggacggcacgctgatctgcaaggtgaagattcgcggcaccaacttcccccccgacggccccgtaatgcagaagaagaccatgggctgggaggcctccaccgagatgctgtaccccaaggacggcgtgctgaagggccatagctatcaggccctgaagctgaaggacggcggccactacctggtggagttcgagaccatctacatggccaagaagcccgtgcaactgcccggcgattactgtgtggacaccaagctggacatcacctcccacaacgaggactacaccatcgtggaacagtacgggcgctccgagggccgccaccgtctgggcatggacgagcggtacaagtaaggatccggctgctaacaaagcccgaaaggaagctgagttggctgctgccaccgctgagcaataactagcataaccccttggggcctctaaacgggtcttgaggggttttttgctgaaaggaggaactatatccggatatcccgcaagaggcccggcagtaccggcataaccaagcctatgcctacagcatccagggtgacggtgccgaggatgacgatgagcgcattgttagatttcatacacggtgcctgactgcgttagcaatttaactgtgataaactaccgcattaaagcttatcgatgataagctgtcaaacatgagaattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactg

SEQ ID NO: 7 - DNA, 12039 bp, Artificial Sequence
  synthetic construct
gtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagcaagctcatggctgactaattttttttatttatgcagaggccgaggccgcctcggcctctgagctattccagaagtagtgaggaggcttttttggaggcctaggcttttgcaaaaagctccccgtggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgacatgattacgaatttcacaaataaagcatttttttcactgcattctagttgtggtttgtccaaactcatcaatgtatcttatcatgtctggatcaactggataactcaagctaaccaaaatcatcccaaacttcccaccccataccctattaccactgccaattacctgtggtttcatttactctaaacctgtgattcctctgaattattttcattttaaagaaattgtatttgttaaatatgtactacaaacttagtagttggaagggctaattcactcccaaagaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgccgaattcacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggactaggatcctttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagctcggtacccgggtcgaggtaggcgtgtacggtgggaggcctatataagcagagctcgtttagtgaaccgtcagatcgcctggagacgccatccacgctgttttgacctccatagaagacaccgggaccgatccagcctccgcggccccgaactagtggccaccatggactacaaggatgacgatgacaagcttgaagcaaccggaaatggtgaatatgcatggtattatgaaggaagaaatgggtggtggcagtacgatgagcgcactagtagagagctggaagatgctttttccaaaggtaaaaagaacactgaaatgttaattgctggctttctgtatgtcgctgatcttgaaaacatggttcaatataggagaaatgaacatggacgtcgcaggaagattaagcgagatataatagatataccaaagaagggagtagctggacttaggctagaattctcgacagggcatggcaccggcagcaccggcagcggcagctccggtaccgcctcctccgaatcgaccggtagttcagtcttcacactcgaagatttcgttggggactgggagcagacagccgcctacaacctggaccaagtccttgaacagggaggtgtgtccagtttgttacagaatctcgcggtgtccgtaactccgatccaaaggattgtccggagcggtgaaaatgcgctgaagatcgacatccatgtcatcatcccgtatgaaggtctgagcgccgaccaaatggcccagatcgaagaggtttttaaggtggtgtaccctgtggatgatcatcactttaaggtgatcctgccctatggcacactggtaatcgacggggttacgccgaacatgctcaactatttcggacggccgtatgaaggcatcgccgtgttcgacggcaaaaagatcactgtaacagggaccctgtggaacggcaacaaaattatcgacgagcgcctgatcacccccgacggctccatgctgttccgagtaaccatcaacagttaaggaagcggagccacgaacttctctctgttaaagcaagcaggagatgttgaagaaaaccccgggcctatggactacaaggatgacgatgacaagcttgaagcaaccggaaatggtgaatatgcatggtattatgaaggaagaaatgggtggtggcagtacgatgagcgcactagtagagagctggaagatgctttttccaaaggtaaaaagaacactgaaatgttaattgctggctttctgtatgtcgctgatcttgaaaacatggttcaatataggagaaatgaacatggacgtcgcaggaagattaagcgagatataatagatataccaaagaagggagtagctggacttaggctagaattctcgacagggcatggcaccggcagcaccggcagcggcagctccggtaccgcctcctccgaatcgaccggtagttcagtgaccggctaccggctgttcgaggagattctggatatctccggaggtatggtgagcaagggcgaggagaataacatggccatcatcaaggagttcatgcgcttcaaggtgcgcatggagggctccgtgaacggccacgagttcgagatcgagggcgagggcgagggccgcccctacgagggctttcagaccgttaagctgaaggtgaccaagggtggccccctgcccttcgcctgggacatcttgtcccctcagttcacctacggctccaaggcctacgtgaagcaccccgccgacatccccgactacctcaagctgtccttccccgagggcttcaagtgggagcgcgtgatgaacttcgaggacggcggcgtggtgaccgtgactcaggactcctccctgcaggacggcgagttcatctacaaggtgaagctgcgcggcaccaacttcccctccgacggccccgtaatgcagaagaagaccatgggcatggaggcctcctccgagcggatgtaccccgaggacggcgccctgaagggcgaggacaagctcaggctgaagctgaaggacggcggccactacacctccgaggtcaagaccacctacaaggccaagaagcccgtgcagttgcccggcgcctacatcgtcgacatcaagttggacatcacctcccacaacgaggactacaccatcgtggaacagtacgaacgcgccgagggccgccactccaccggcggcatggacgagctgtacaagtaagtagtaatgattaattaaactagaaattctaccgggtaggggaggcgcttttcccaaggcagtctggagtaacccacccaagatctggcctccgcgccgggttttggcgcctcccgcgggcgcccccctcctcacggcgagcgctgccacgtcagacgaagggcgcagcgagcgtcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgattatataaggacgcgccgggtgtggcacagctagttccgtcgcagccgggatttgggtcgcggttcttgtttgtggatcgctgtgatcgtcacttggtgagtagcgggctgctgggctggccggggctttcgtggccgccgggccgctcggtgggacggaagcgtgtggagagaccgccaagggctgtagtctgggtccgcgagcaaggttgccctgaactgggggttggggggagcgcagcaaaatggcggctgttcccgagtcttgaatggaagacgcttgtgaggcgggctgtgaggtcgttgaaacaaggtggggggcatggtgggcggcaagaacccaaggtcttgaggccttcgctaatgcgggaaagctcttattcgggtgagatgggctggggcaccatctggggaccctgacgtgaagtttgtcactgactggagaactcggtttgtcgtctgttgcgggggcggcagttatggcggtgccgttgggcagtgcacccgtacctttgggagcgcgcgccctcgtcgtgtcgtgacgtcacccgttctgttggcttataatgcagggtggggccacctgccggtaggtgtgcggtaggcttttctccgtcgcaggacgcagggttcgggcctagggtaggctctcctgaatcgacaggcgccggacctctggtgaggggagggataagtgaggcgtcagtttctttggtcggttttatgtacctatcttcttaagtagctgaagctccggttttgaactatgcgctcggggttggcgagtgtgttttgtgaagttttttaggcaccttttgaaatgtaatcatttgggtcaatatgtaattttcagtgttagactagtaaattgtccgctaaattctggccgtttttggcttttttgttagacgaagcttggtaccgagctcggatctccaccccgtaccggtcctgcagtcgaattcaccatgtctagactggacaagagcaaagtcataaacggagctctggaattactcaatggtgtcggtatcgaaggcctgacgacaaggaaactcgctcaaaagctgggagttgagcagcctaccctgtactggcacgtgaagaacaagcgggccctgctcgatgccctgccaatcgagatgctggacaggcatcatacccacttctgccccctggaaggcgagtcatggcaagactttctgcggaacaacgccaagtcataccgctgtgctctcctctcacatcgcgacggggctaaagtgcatctcggcacccgcccaacagagaaacagtacgaaaccctggaaaatcagctcgcgttcctgtgtcagcaaggcttctccctggagaacgcactgtacgctctgtccgccgtgggccactttacactgggctgcgtattggaggaacaggagcatcaagtagcaaaagaggaaagagagacacctaccaccgattctatgcccccacttctgagacaagcaattgagctgttcgaccggcagggagccgaacctgccttccttttcggcctggaactaatcatatgtggcctggagaaacagctaaagtgcgaaagcggcgggccgaccgacgcccttgacgattttgacttagacatgctcccagccgatgcccttgacgactttgaccttgatatgctgcctgctgacgctcttgacgattttgaccttgacatgctccccgggtaactaagtaaggatccgcggccgcactagaggaattccgcccctctccctcccccccccctaacgttactggccgaagccgcttggaataaggccggtgtgtgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtagtcaacaaggggctgaaggatgcccagaaggtaccccattgtatgggaatctgatctggggcctcggtgcacatgctttacatgtgtttagtcgaggttaaaaaaacgtctaggccccccgaaccacggggacgtggttttcctttgaaaaacacgatgataagcttaccggtccaccatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgatgtacaagtaaagcggccgcgactctagatcataatcagccataccacatttgtagaggttttacttgctttaaaaaacctcccacacctccccctgaacctgaaacataaaatgaatgcaattgttgttgtttagtccctcccaattcgatatcaagcttatcgatcgatagatcctaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctgagatcctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggcccgggttaattaaggaaagggctagatcattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctg

SEQ ID NO: 8 - DNA, 600 bp, Archaeoglobus fulgidus
atggaacggcgtactttaatcatggaggtgctttttgaggccaaggttggggacatcactctgaagctcgctcagggggacatcacccaatacccggcaaaggcaattgtcaacgcggccaacaagaggctggagcacggcggaggggtggcttatgccatcgcaaaagcgtgtgcaggagatgccgggctctacacggaaatcagcaaaaaggccatgagagagcagtttggaagagactacatcgaccacggcgaggtcgttgttacaccggccatgaacctcgaggagaggggaataaagtacgttttccacaccgtggggccgatatgcagcggcatgtggagcgaagaactgaaagagaagctttacaaggcttttcttggcccgctggagaaggcggaggagatgggcgtcgaatccatagccttccctgctgtgagcgctgggatatacggctgtgatctggaaaaggttgttgagacgttcctcgaagctgtgaagaacttcaagggttcggctgtcaaggaagtcgcgcttgtaatctacgacagaaagtctgcggaggtggcgctgaaggtctttgagaggagtctttga

SEQ ID NO: 9 - DNA, 255 bp, Homo sapiens
atgggaaatggtgaatatgcatggtattatgaaggaagaaatgggtggtggcagtacgatgagcgcactagtagagagctggaagatgctttttccaaaggtaaaaagaacactgaaatgttaattgctggctttctgtatgtcgctgatcttgaaaacatggttcaatataggagaaatgaacatggacgtcgcaggaagattaagcgagatataatagatataccaaagaagggagtagctggacttaggtaa

SEQ ID NO: 10 - DNA, 252 bp, Homo sapiens
atggattcagttctacaaggttctgaaggaaacaaggtcaagaggacatcctgcatgtatggggcaaactgctataggaagaatcctgttcattttcaacattttagccatcctggtgatagtgattatggaggtgtacaaatcgtgggccaagatgagactgatgaccggcctgaatgtccctatggaccatcctgttataggaagaatccccagcacaagatagaatatagacataatacgcttccatga

SEQ ID NO: 11 - protein, 349 aa, Artificial Sequence
  synthetic construct
DYKDDDDKLEATGNGEYAWYYEGRNGWWQYDERTSRELEDAFSKGKKNTEMLIAGFLYVADLENMVQYRRNEHGRRRKIKRDIIDIPKKGVAGLRLEFSTGHGTGSTGSGSSGTASSESTGSSMASKSEEVIKEFMRFKVRLEGSMNGHEFEIEGEGEGRPYEGTQTAKLKVTKGGPLPFAWDILSPLIMYGSKMYVKHPADVPDYMKLSFPEGFKWERVMHFEDGGLVTATQDSSLQDGTLIYKVKMRGTNFPPDGPVMQKKTLGWDYATERLYPEEGVLKGELLGRLKLKDGGLNLVESKTIYMAKKPVQLPGYYFVDTKLDITSHNEDYTIVEQYERSEGRHHLGH

SEQ ID NO: 12 - protein, 349 aa, Artificial Sequence
  synthetic construct
MYPYDVPDYALEATGNGEYAWYYEGRNGWWQYDERTSRELEDAFSKGKKNTEMLIAGFLYVADLENMVQYRRNEHGRRRKIKRDIIDIPKKGVAGLRLEFSTGHGTGSTGSGSSGTASSESTGSSTIKEFMRFKVRMEGSMNGHEFEIEGEGEGRPYEGTQTAKLKVTKGGPLPFAWDILSPQIMYGSKAYVRHPADIPDYKKLPFPEGFKWERVMNFEDGGLVTVTQDSSLQDGTLICKVKIRGTNFPPDGPVMQKKTMGWEASTEMLYPKDGVLKGHSYQALKLKDGGHYLVEFETIYMAKKPVQLPGDYCVDTKLDITSHNEDYTIVEQYGRSEGRHRLGMDERYK

SEQ ID NO: 13 - protein, 363 aa, Artificial Sequence
  synthetic construct
MGHHHHHHHHHHSSGHIDDDDKHMLEGNGEYAWYYEGRNGWWQYDERTSRELEDAFSKGKKNTEMLIAGFLYVADLENMVQYRRNEHGRRRKIKRDIIDIPKKGVAGLRLEFSTGHGTGSTGSGSSGTASSESTGSSMASKSEEVIKEFMRFKVRLEGSMNGHEFEIEGEGEGRPYEGTQTAKLKVTKGGPLPFAWDILSPLIMYGSKMYVKHPADVPDYMKLSFPEGFKWERVMHFEDGGLVTATQDSSLQDGTLIYKVKMRGTNFPPDGPVMQKKTLGWDYATERLYPEEGVLKGELLGRLKLKDGGLNLVESKTIYMAKKPVQLPGYYFVDTKLDITSHNEDYTIVEQYERSEGRHHLGH

SEQ ID NO: 14 - protein, 361 aa, Artificial Sequence
  synthetic construct
MGHHHHHHHHHHSSGHIDDDDKHMLEGNGEYAWYYEGRNGWWQYDERTSRELEDAFSKGKKNTEMLIAGFLYVADLENMVQYRRNEHGRRRKIKRDIIDIPKKGVAGLRLEFSTGHGTGSTGSGSSGTASSESTGSSTIKEFMRFKVRMEGSMNGHEFEIEGEGEGRPYEGTQTAKLKVTKGGPLPFAWDILSPQIMYGSKAYVRHPADIPDYKKLPFPEGFKWERVMNFEDGGLVTVTQDSSLQDGTLICKVKIRGTNFPPDGPVMQKKTMGWEASTEMLYPKDGVLKGHSYQALKLKDGGHYLVEFETIYMAKKPVQLPGDYCVDTKLDITSHNEDYTIVEQYGRSEGRHRLGMDERYK

SEQ ID NO: 15 - protein, 282 aa, Artificial Sequence
  synthetic construct
MDYKDDDDKLEATGNGEYAWYYEGRNGWWQYDERTSRELEDAFSKGKKNTEMLIAGFLYVADLENMVQYRRNEHGRRRKIKRDIIDIPKKGVAGLRLEFSTGHGTGSTGSGSSGTASSESTGSSVFTLEDFVGDWEQTAAYNLDQVLEQGGVSSLLQNLAVSVTPIQRIVRSGENALKIDIHVIIPYEGLSADQMAQIEEVFKVVYPVDDHHFKVILPYGTLVIDGVTPNMLNYFGRPYEGIAVFDGKKITVTGTLWNGNKIIDERLITPDGSMLFRVTINS

SEQ ID NO: 16 - protein, 376 aa, Artificial Sequence
  synthetic construct
MDYKDDDDKLEATGNGEYAWYYEGRNGWWQYDERTSRELEDAFSKGKKNTEMLIAGFLYVADLENMVQYRRNEHGRRRKIKRDIIDIPKKGVAGLRLEFSTGHGTGSTGSGSSGTASSESTGSSVTGYRLFEEILDISGGMVSKGEENNMAIIKEFMRFKVRMEGSVNGHEFEIEGEGEGRPYEGFQTVKLKVTKGGPLPFAWDILSPQFTYGSKAYVKHPADIPDYLKLSFPEGFKWERVMNFEDGGVVTVTQDSSLQDGEFIYKVKLRGTNFPSDGPVMQKKTMGMEASSERMYPEDGALKGEDKLRLKLKDGGHYTSEVKTTYKAKKPVQLPGAYIVDIKLDITSHNEDYTIVEQYERAEGRHSTGGMDELYK

SEQ ID NO: 17 - DNA, 38 bp, Artificial Sequence
  synthetic construct - primer
aggggcggaattcctctagttcaatgccccaggtggtg

SEQ ID NO: 18 - DNA, 38 bp, Artificial Sequence; identical to SEQ ID NO: 17
  synthetic construct - primer

SEQ ID NO: 19 - DNA, 45 bp, Artificial Sequence
  synthetic construct - primer
attacgctcttgaagcaaccatggccaccatcaaagagttcatgc

SEQ ID NO: 20 - DNA, 38 bp, Artificial Sequence
  synthetic construct - primer
tagggccctctagatgcatgttacttgtaccgctcgtc

SEQ ID NO: 21 - DNA, 48 bp, Artificial Sequence
  synthetic construct - primer
atgacaagcttgaagcaaccggaaatggtgaatatgcatggtattatg

SEQ ID NO: 22 - DNA, 38 bp, Artificial Sequence; identical to SEQ ID NO: 17
  synthetic construct - primer

SEQ ID NO: 23 - DNA, 40 bp, Artificial Sequence
  synthetic construct - primer
attacgctcttgaagcaaccggaaatggtgaatatgcatg

SEQ ID NO: 24 - DNA, 38 bp, Artificial Sequence; identical to SEQ ID NO: 20
  synthetic construct - primer

SEQ ID NO: 25 - DNA, 49 bp, Artificial Sequence
  synthetic construct - primer
tatcgacgacgacgacaagcatatgctcgagatggcgagcaagagcgag

SEQ ID NO: 26 - DNA, 43 bp, Artificial Sequence
  synthetic construct - primer
tcgggctttgttagcagccggatcctcaatgccccaggtggtg

SEQ ID NO: 27 - DNA, 50 bp, Artificial Sequence
  synthetic construct - primer
tatcgacgacgacgacaagcatatgctcgagaccatcaaagagttcatgc

SEQ ID NO: 28 - DNA, 43 bp, Artificial Sequence
  synthetic construct - primer
tcgggctttgttagcagccggatccttacttgtaccgctcgtc

SEQ ID NO: 29 - DNA, 51 bp, Artificial Sequence
  synthetic construct - primer
tatcgacgacgacgacaagcatatgctcgagggaaatggtgaatatgcatg

SEQ ID NO: 30 - DNA, 43 bp, Artificial Sequence; identical to SEQ ID NO: 26
  synthetic construct - primer

SEQ ID NO: 31 - DNA, 51 bp, Artificial Sequence; identical to SEQ ID NO: 29
  synthetic construct - primer

SEQ ID NO: 32 - DNA, 43 bp, Artificial Sequence; identical to SEQ ID NO: 28
  synthetic construct - primer

SEQ ID NO: 33 - DNA, 51 bp, Artificial Sequence
  synthetic construct - primer
tatcgacgacgacgacaagcatatgctcgagggtgaagtcagtaaggcagc

SEQ ID NO: 34 - DNA, 28 bp, Artificial Sequence
  synthetic construct - primer
agaattctaggttggcgtccagcttggc

SEQ ID NO: 35 - DNA, 32 bp, Artificial Sequence
  synthetic construct - primer
ggacgccaacctagaattctcgacagggcatg

SEQ ID NO: 36 - DNA, 43 bp, Artificial Sequence; identical to SEQ ID NO: 26
  synthetic construct - primer

SEQ ID NO: 37 - DNA, 28 bp, Artificial Sequence
  synthetic construct - primer
ggacgccaacctagaattctcgacaggg

SEQ ID NO: 38 - DNA, 43 bp, Artificial Sequence; identical to SEQ ID NO: 28
  synthetic construct - primer

SEQ ID NO: 39 - DNA, 51 bp, Artificial Sequence
  synthetic construct - primer
tatcgacgacgacgacaagcatatgctcgaggattcagttctacaaggttc

SEQ ID NO: 40 - DNA, 34 bp, Artificial Sequence
  synthetic construct - primer
agaattctagtggaagcgtattatgtctatattc

SEQ ID NO: 41 - DNA, 32 bp, Artificial Sequence
  synthetic construct - primer
tacgcttccactagaattctcgacagggcatg

SEQ ID NO: 42 - DNA, 43 bp, Artificial Sequence; identical to SEQ ID NO: 26
  synthetic construct - primer

SEQ ID NO: 43 - DNA, 28 bp, Artificial Sequence
  synthetic construct - primer
tacgcttccactagaattctcgacaggg

SEQ ID NO: 44 - DNA, 43 bp, Artificial Sequence; identical to SEQ ID NO: 28
  synthetic construct - primer

SEQ ID NO: 45 - DNA, 52 bp, Artificial Sequence
  synthetic construct - primer
tatcgacgacgacgacaagcatatgctcgagatggaacggcgtactttaatc

SEQ ID NO: 46 - DNA, 28 bp, Artificial Sequence
  synthetic construct - primer
agaattctagaagactcctctcaaagac

SEQ ID NO: 47 - DNA, 32 bp, Artificial Sequence
  synthetic construct - primer
gaggagtcttctagaattctcgacagggcatg

SEQ ID NO: 48 - DNA, 43 bp, Artificial Sequence; identical to SEQ ID NO: 26
  synthetic construct - primer

SEQ ID NO: 49 - DNA, 28 bp, Artificial Sequence
  synthetic construct - primer
gaggagtcttctagaattctcgacaggg

SEQ ID NO: 50 - DNA, 43 bp, Artificial Sequence; identical to SEQ ID NO: 28
  synthetic construct - primer

SEQ ID NO: 51 - DNA, 38 bp, Artificial Sequence
  synthetic construct - primer
tccgcggccccgaactagtggccaccatggactacaag

SEQ ID NO: 52 - DNA, 41 bp, Artificial Sequence
  synthetic construct - primer
agaggggcggaattcctctagtcttacttgtaccgctcgtc

SEQ ID NO: 53 - DNA, 89 bp, Artificial Sequence
  synthetic construct - primer
tccgcggccccgaactagtggccaccatggactacaaggatgacgatgacaagcttgaagcaaccatggaacggcgtactttaatcatg

SEQ ID NO: 54 - DNA, 28 bp, Artificial Sequence
  synthetic construct - primer
ttcctctagttcaatgccccaggtggtg

SEQ ID NO: 55 - DNA, 28 bp, Artificial Sequence
  synthetic construct - primer
ggggcattgaactagaggaattccgccc

SEQ ID NO: 56 - DNA, 41 bp, Artificial Sequence; identical to SEQ ID NO: 52
  synthetic construct - primer

SEQ ID NO: 57 - DNA, 38 bp, Artificial Sequence
  synthetic construct - primer
caagcttggtaccgagctcggccaccatggactacaag

SEQ ID NO: 58 - DNA, 28 bp, Artificial Sequence
  synthetic construct - primer
ccatggatcctgaactaccggtcgattc

SEQ ID NO: 59 - DNA, 28 bp, Artificial Sequence
  synthetic construct - primer
cggtagttcaggatccatggaagacgcc

SEQ ID NO: 60 - DNA, 44 bp, Artificial Sequence
  synthetic construct - primer
agggccctctagatgcatgctcacataatcataggtcctctgac

SEQ ID NO: 61 - DNA, 38 bp, Artificial Sequence; identical to SEQ ID NO: 57
  synthetic construct - primer

SEQ ID NO: 62 - DNA, 28 bp, Artificial Sequence
  synthetic construct - primer
gtccggatcctgaactaccggtcgattc

SEQ ID NO: 63 - DNA, 31 bp, Artificial Sequence
  synthetic construct - primer
cggtagttcaggatccggacctatgattatg

SEQ ID NO: 64 - DNA, 39 bp, Artificial Sequence
  synthetic construct - primer
agggccctctagatgcatgcttacaatttggactttccg

SEQ ID NO: 65 - DNA, 38 bp, Artificial Sequence
  synthetic construct - primer
tccgcggccccgaactagtgatggactacaaggatgac

SEQ ID NO: 66 - DNA, 28 bp, Artificial Sequence
  synthetic construct - primer
ctccgcttccactgttgatggttactcg

SEQ ID NO: 67 - DNA, 28 bp, Artificial Sequence
  synthetic construct - primer
catcaacagtggaagcggagccacgaac

SEQ ID NO: 68 - DNA, 43 bp, Artificial Sequence
  synthetic construct - primer
gtttaattaatcattactacttacttgtacagctcgtccatgc

(19) United States
(12) Patent Application Publication
(10) Pub. No.: US 2024/0252689 A1
(43) Pub. Date: Aug. 1, 2024

(54) POLY-ADP RIBOSE (PAR) TRACKER OPTIMIZED SPLIT-PROTEIN REASSEMBLY PAR DETECTION REAGENTS

(71) Applicant: THE BOARD OF REGENTS OF THE UNIVERSITY OF TEXAS SYSTEM, Austin, TX (US)

(72) Inventors: W. Lee KRAUS, Dallas, TX (US); Keun Woo RYU, Dallas, TX (US); Sridevi CHALLA, Dallas, TX (US)

(73) Assignee: THE BOARD OF REGENTS OF THE UNIVERSITY OF TEXAS SYSTEM, Austin, TX (US)

(21) Appl. No.: 18/561,044

(22) PCT Filed: May 18, 2022
(86) PCT No.: PCT/US2022/029802

§ 371 Date: Nov. 15, 2023

Related U.S. Application Data
(60) Provisional application No. 63/190,031, filed on May 18, 2021.

(51) Int. Cl. A61K 49/00; C12N 9/10; C12Q 1/48

(52) U.S. Cl. CPC A61K 49/0047; A61K 49/0008; A61K 49/0045; A61K 49/0056; C12N 9/1077; C12Q 1/48; C12Y 204/0203; G01N 2333/91142

(57) ABSTRACT

Provided herein are split reporter systems for detecting poly-ADP ribose polymerase activity in living systems. In some aspects, the split reporter systems comprise a first fusion protein comprising a first fragment of a reporter protein functionally linked to a first poly-ADP ribose binding moiety; and a second fusion protein comprising a second fragment of the reporter protein functionally linked to a second poly-ADP ribose binding moiety wherein the first and second fragments of the reporter protein are each non-functional and capable of recombining, optionally in the presence of a substrate, to form a functional reporter protein capable of producing a detectable signal. Also provided are methods of use thereof.

### CROSS-REFERENCE TO RELATED APPLICATIONS

This application claims the benefit of, and priority to U.S. Provisional Application No. 63/190,031, filed May 18, 2021, the entire contents of which are hereby incorporated by reference in their entirety.

### ACKNOWLEDGEMENT OF GOVERNMENT SUPPORT

This invention was made with government support under Grant No. DK069710 awarded by the National Institutes of Health. The government has certain rights in the invention.

### SEQUENCE LISTING

The instant application contains a Sequence Listing which has been submitted electronically in ASCII format and is hereby incorporated by reference in its entirety. Said ASCII copy, created on May 17, 2021, is named UTSD3881_SEQUENCELISTING.txt and is 84,500 bytes in size.

### BACKGROUND

### 1. Field of the Invention

The present disclosure is generally directed to fusion proteins and their use for detecting poly-ADP-ribose polymerase (PARP) activity.

### 2. Discussion of Related Art

ADP-ribosylation (ADPRylation) is a regulatory post-translational modification (PTM) of proteins on a range of amino acid residues (including Asp, Glu, Ser, Cys, Lys, Arg) that results in the reversible attachment of ADP-ribose (ADPR) subunits on substrate proteins, acting to control their functions through a variety of mechanisms. Members of the PARP family of enzymes plays a key role in catalyzing cellular ADPRylation. The mammalian PARP family contains 17 members, each possessing an ADP-ribosyltransferase catalytic domain that is functionalized with other domains that confer additional biochemical functions or direct the proteins to specific cellular compartments. While mono(ADP-ribosyl) transferases (MARTs) (PARP ‘monoenzymes’) modify their target proteins by the addition of a single ADPR moiety [i.e., addition of mono(ADP-ribose) via MARylation], PARP ‘polyenzymes’ catalyze the formation of branched or linear chains of multiple ADPR moieties (i.e., addition of poly(ADP-ribose) via PARylation). PARP enzymes are active in DNA repair pathways and are upregulated after DNA damage. Accordingly, detecting their activity can be beneficial in conditions characterized by elevated DNA damage like cancer.

### BRIEF SUMMARY

In accordance with an aspect of the disclosure, provided is a split reporter system for detecting poly-ADP ribose polymerase (PARP) activity comprising: (a) a first fusion protein comprising a first fragment of a reporter protein functionally linked to a first poly-ADP ribose binding moiety; and (b) a second fusion protein comprising a second fragment of the reporter protein functionally linked to a second poly-ADP ribose binding moiety; wherein the first and second fragments of the reporter protein are each non-functional and capable of recombining, optionally in the presence of a substrate, to form a functional reporter protein capable of producing a detectable signal.

In another aspect of the disclosure, provided is another split reporter system for detecting poly-ADP ribose polymerase (PARP) activity comprising: (a) a first fusion protein comprising a first monomer of a dimerization-dependent reporter system functionally linked to a first poly-ADP ribose binding moiety; and (b) a second fusion protein comprising a second monomer of the dimerization dependent reporter system functionally linked to a second poly-ADP ribose binding moiety; wherein the first and second monomers of the dimerization dependent reporter system are capable of combining to form a heterodimer of the dimerization-dependent reporter system, the heterodimer capable of emitting a detectable light signal.

Also provided are nucleic acid constructs, expression vectors and host cells that can express the fusion proteins described herein.

Also provided is a method of detecting poly-ADP ribose polymerase (PARP) activity in a cell or tissue suspected of having PARP activity, the method comprising: (a) introducing the first and second fusion proteins described herein into the cell or tissue; (b) maintaining the cell or tissue for a time and under conditions sufficient for the first and second fusion proteins to bind to one or more poly-ADP ribose (PAR) chains and combine to produce a signal; and (c) detecting the signal, wherein the signal is proportional to the PARP activity in the system.

Also provided is a method of assessing the efficacy of a potential therapeutic, the method comprising: (a) introducing the first and second fusion proteins described herein into a cell or tissue; (b) applying the potential therapeutic to the cell or tissue; (c) maintaining the cell or tissue for a time and under conditions sufficient for the first and second fusion proteins to bind to one or more poly-ADP ribose (PAR) chains and combine to produce a signal; and (d) detecting the signal, wherein the signal is indicative of the efficacy of the potential therapeutic.

Also provided are kits having compositions disclosed herein and for use in methods disclosed herein.

### BRIEF DESCRIPTION OF THE DRAWINGS

FIG. 1A shows a schematic diagram of an exemplary PAR-dependent fluorescent tracker system.

FIG. 1B is a schematic diagram of a genetic construct used to express an exemplary fluorescent based PAR tracker (top) and an annotated poly-ADP chain chemical structure (bottom) with preferred binding epitopes for representative PAR binding domains labeled.

FIG. 1C is an illustrative bar graph plotting relative fluorescence detected in in vitro ADP ribosylation assays using various PAR-dependent trackers.

FIG. 1D is a representative immunoblot showing a time course of PAR formation using recombinant PARP in vitro.

FIG. 1E is a representative graph plotting relative fluorescence measured from various PAR-trackers during PAR formation in vitro.

FIG. 1F is a representative immunoblot showing a time course of in vitro PAR degradation using recombinant PARP in vitro.

FIG. 1G is a representative graph plotting relative fluorescence measured from various PAR-trackers during PAR degradation in vitro.

FIGS. 2A and 2B provide illustrative immunofluorescence images of (2A) and relative fluorescent intensity measured in (2B) 293T cells expressing exemplary PAR trackers or control fluorescent monomers in the presence or absence a DNA damaging agent and in the presence or absence of a PARP inhibitor.

FIGS. 2C and 2D provide a time course live cell imaging of (2C) and relative fluorescent intensity measured in (2D) Hela cells expressing exemplary PAR trackers or control fluorescent monomers in the presence or absence of a DNA damaging agent and in the presence or absence of a PARP inhibitor.

FIG. 3A is a schematic diagram of plasmid constructs used to express an exemplary luminescent based PAR tracker in mammalian cells.

FIGS. 3B and 3C show representative bioluminescent images (3B) and quantified relative luminescence (3C) of MDA-MB-231-luc cells subjected to dox induced expression of an exemplary luminescent based PAR tracker in the presence or absence of a PARP inhibitor or a PARG inhibitor.

FIG. 3D provides an exemplary western blot of cell lysates obtained from a cell line subjected to siRNA mediated knockdown of PARP1 or PARP2.

FIGS. 3E and 3F show representative bioluminescent images (3E) and quantified relative luminescence (3F) measured from an exemplary luminescent based PAR tracker and a control luciferase in a cell line in the presence or absence of siRNA mediated knockdown of PARP1 or PARP2.

FIG. 4A shows a representative immunoblot showing PAR levels in a cell line treated with a PARP inhibitor or a PARG inhibitor prior to UV radiation.

FIGS. 4B to 4D show representative bioluminescent images (4B) and quantified bioluminescence (4C and 4D) measured from an exemplary luminescent based PAR tracker (4C) and a control luciferase (4D) in a cell line in the presence or absence of a PARP inhibitor or a PARG inhibitor before and after UV irradiation.

FIG. 4E shows a time course of bioluminescence imaging of 231-PAR-T NanoLuc and 231-Full Nano luciferase cells treated with 20 UM Niraparib or 20 μM PARG inhibitor for 2 hr prior to UV radiation. Each point in the graph represents the mean±SEM of the relative levels of luminescence from PAR-T NanoLuc normalized to full Nano luciferase (n=3).

FIG. 5A provides a schematic diagram of a gene construct for expressing an exemplary luminescent based PAR tracker and a control luciferase in a cell line before injection into a mouse and detection of PAR tracker luminescence in vivo.

FIGS. 5B and 5C show representative bioluminescent images (5B) and quantification of relative luminescence (5C) of tumors formed in mice injected with a cell line expressing an exemplary luminescent based PAR tracker following administration of a vehicle, a PARP inhibitor or a PARG inhibitor to the mouse.

FIGS. 5D to 5E show analysis of a time course of bioluminescence imaging of 231-PAR-T NanoLuc cells transplanted into mice and treated with PARG inhibitor. Bioluminescence imaging (5D) with the region of interest expanded (5E). Each point in the graph in (5F) represents the mean±SEM of the relative levels of luminescence of NanoLuc of PAR-T NanoLuc (n=4).

FIGS. 6A to 6C provide an illustrative Coomassie blue stain of recombinant fluorescent-based trackers conjugated to various ADPR binding domains (6A) and an illustrative Coomassie blue stain (6B) and immunoblot (6C) of recombinant PARP-1 and PARP-3 proteins used herein.

FIGS. 6D and 6E provide representative fluorescent measurements (6D) and a heatmap (6E) of in vitro PARylation assays performed using various PAR-binding domains.

FIGS. 6F and 6G provide a representative immunoblot (S1F) and fluorescent measurements (S1G) of in vitro PAR formation using recombinant PARP-1 and indicated concentrations of NAD+.

FIG. 6H provides an illustrative graph showing fluorescent measurements of in vitro PAR degradation as measured by various fluorescent based PAR trackers.

FIGS. 6I and 6J provide a representative immunoblot and fluorescent measurements of PARylation in a cell lysate system as detected using exemplary fluorescent based PAR trackers.

FIG. 7A provides a schematic diagram of plasmid constructs used to express an exemplary fluorescent based PAR tracker in mammalian cells.

FIGS. 7B and 7C provides illustrative immunofluorescent images and quantification of an exemplary fluorescent based PAR tracker expressed in mammalian cells in the presence of a DNA damaging agent (H2O2).

FIGS. 8A and 8B provides illustrative confocal images (8A) and quantification (8B) of cancer spheroids formed using a transgenic cell line subjected to dox-induced expression of an exemplary fluorescent based PAR tracker in the presence or absence of a PARP inhibitor. Nuclei are labeled in red (mCherry).

FIGS. 8C and 8D provides representative images (8C) and quantification (8D) of Z-projections of cancer spheroids formed using MCF-7 cells subjected to Dox-induced expression of the PAR-T ddGFP. The spheroids were treated with 20 UM Niraparib and live-cell imaging was performed at the indicated times. (Left) The spheroids were divided into ‘outer’ and ‘core’ sections for quantification as indicated by the white circles. (Right) Enlargement of the indicated areas from the left panels (yellow, core; pink, outer) as indicated. Each point in the graph in (D) represents the mean±SEM of the relative levels of PAR-T ddGFP fluorescence intensity normalized to mCherry (n=5, one-way ANOVA, *p<0.05 and **p<0.01).

FIG. 9A provides a representative graph plotting bioluminescence detected in cell lysates prepared from HEK293T cells expressing the indicated exemplary luminescent based PAR trackers after treatment with a vehicle or a PARP inhibitor.

FIG. 9B provides an exemplary immunoblot showing PAR levels in cell lysates from HEK293T cells exposed to a PARP inhibitor or a PARG inhibitor.

FIG. 9C provides representative bioluminescent images of HEK293T cells expressing exemplary split firefly luciferase based PAR trackers.

FIGS. 9D and 9E provides representative bioluminescent images and quantification of Hela cells expressing split firefly luciferase based PAR trackers in the presence or absence of a PARP inhibitor or a PARG inhibitor.

FIGS. 10A and 10B provides representative bioluminescence imaging (10A) and relative levels of the ratio of luminescence of Nano luciferase to firefly luciferase (10B) of an indicated number of 231-PAR-T Nluc cells.

FIG. 11A provides quantitative analysis of Western blot analysis and fluorescence measurements (shown in FIG. 1G) of the time course of in vitro PAR degradation using recombinant ARH3. Each line plot in the graph represents mean±SEM of relative intensities (n=3).

FIG. 11B provides quantitative analysis of Western blot analysis and bioluminescence imaging (shown in FIG. 4B) of 231-PAR-T NanoLuc cells treated with 20 μM Niraparib or 20 μM PARG inhibitor for 2 hr prior to UV radiation. Each bar in the graph represents the mean±SEM of the relative intensities (n=3, one-way ANOVA, *p<0.05, **p<0.001, and ***p<0.0001; ns=not significant).

FIG. 11C provides measurements of ELISA and fluorescence intensities using 0, 0.625, 1.25, and 2.5 nM concentrations of purified PAR. Each bar in the graph in represents the mean±SEM of the relative intensities (n=3, paired t-test, *p<0.05, **p<0.01, and ***p<0.001; ns=not significant).

FIGS. 11D and 11E provides an immunofluorescence assay (11D) and quantification (11E) using WWE-Fc to measure PAR formation in response to H2O2 using 293T cells. The cells were treated with 20 μM PJ34 (vs. untreated control, ‘Un’) for 2 hr prior to 15 min of treatment with 1 mM H₂O₂. The images were collected using a confocal microscope. Each bar in the graph in (11E) represents the mean±SEM of the relative levels of the fluorescence intensity of PAR normalized to DAPI (n=3 biological replicates with at least 150 cells in total, one-way ANOVA, ***p<0.0001).

FIG. 11F provides a representation of the dynamic ranges of PAR-T sensors in comparison to other available PAR detection tools as indicated: (a) Western blotting with WWE-Fc versus live-cell luciferase assay using PAR-T NanoLuc was performed using UV-induced DNA damage in MDA-MB-231 Luc cells (from (FIG. 11B)); (b) Immunofluorescence with WWE-Fc versus live-cell imaging using PAR-T ddGFP was performed using H2O2-mediated PARP-1 activation in 293T cells (from (FIG. 11D)); (c) Western blotting with WWE-Fc versus fluorescence assay with PAR-T ddGFP was performed using ARH3 mediated degradation of PAR in vitro (from (FIG. 11A)); (d) ELISA versus fluorescence assay with PAR-T ddGFP was performed using immobilized PAR (from FIG. 11C).

FIG. 12A and FIG. 12B provides bioluminescence imaging of PAR-T NanoLuc (12A) and unsplit NanoLuc (12B) in 3T3-L1 cells subjected to adipogenic differentiation for 12 or 24 hr.

FIGS. 12C and 12D provide quantification of signals from PAR-T NanoLuc (12C) and unsplit NanoLuc (12D) during adipogenesis. Each bar in the graph represents the mean±SEM of the relative levels of the luminescence of NanoLuc (n=4; t-test, **p<0.01 and ***p<0.001). Comparisons between experimental conditions with the intact NanoLuc are not statistically significant (ns).

### DETAILED DESCRIPTION

Provided herein are PAR trackers comprising a set of optimized split protein reassembly poly(ADP-ribose) (PAR) detection reagents. In general embodiments, the system comprises a first fusion protein and a second fusion protein. Each fusion protein can comprise (a) a first or a second poly-ADP ribose (PAR) binding moiety and (b) a first or second non-functional fragment of a reporter protein, wherein the first and second fragments of the reporter protein are each non-functional but can recombine, optionally in the presence of a substrate, to form a functional reporter protein capable of producing a detectable signal. In further embodiments, the first or second non-functional fragments of the reporter protein can be replaced with full monomers of a dimerization dependent reporter system, wherein the monomers are each non-functional (or quenched) but can recombine, optionally in the presence of a substrate, to form a heterodimer capable of producing a detectable signal.

The PAR trackers described herein provide significant improvements compared to previously reported split protein reassembly reagents. Namely, the PAR trackers have a higher sensitivity and affinity for ADP-ribose, and allow for real time assessment of dynamic PAR production in extracts, living cells and living mammals. The PAR trackers provided allow for enhanced detection and measurement of PAR production and levels in a variety of systems in a manner not achieved by other available tools.

### I. Fusion Proteins

In various aspects, the split reporter system for detecting PARP activity comprises a first fusion protein and a second fusion protein. The fusion proteins each comprise a fragment of a reporter protein functionally linked to a poly-ADP ribose binding moiety. Alternatively, the fusion proteins can comprise a monomer of a dimerization dependent fluorescent protein functionally linked to a poly-ADP ribose binding moiety.

As used herein “functionally linked” refers to a peptide connection (direct or indirect) that covalently links the two components (e.g., reporter protein fragment, a monomer of a dimerization dependent fluorescent protein, and/or the poly-ADP ribose binding moiety) in a single fusion protein. In various embodiments, the two components are directly linked. In other embodiments, the two components are indirectly linked (e.g., through a peptide linker).

### 1. Reporter Proteins

In various aspects, the fusion proteins comprise a fragment of a reporter protein. For example, the fragment of the reporter protein can comprise a fragment of a fluorescent protein (i.e., a split-GFP, a split-RFP, a split-YFP). In other aspects, the fragment of the reporter protein can comprise a luminescent protein.

In various embodiments, the complementary set of fragments or proteins can comprise a fluorescent-based reporter. Non limiting examples of fluorescent proteins include green fluorescent proteins (e.g., GFP, GFP-2, tagGFP, turboGFP, EGFP, Emerald, Azami Green, Monomeric Azami Green, CopGFP, AceGFP, ZsGreenI), yellow fluorescent proteins (e.g., YFP, EYFP, Citrine, Venus, YPet, PhiYFP, ZsYellowI), blue fluorescent proteins (e.g., EBFP, EBFP2, Azurite, mKalamal, GFPuv, Sapphire, T-sapphire,), cyan fluorescent proteins (e.g. ECFP, Cerulean, CyPet, AmCyanI, Midoriishi-Cyan), red fluorescent proteins (e.g., mKate, mKate2, mPlum, DsRed monomer, mCherry, mRFP1, DsRed-Express, DsRed2, DsRed-Monomer, HcRed-Tandem, HcRedI, AsRed2, eqFP61 1, mRasberry, mStrawberry, Jred), and orange fluorescent proteins (e.g., mOrange, mKO, Kusabira-Orange, Monomeric Kusabira-Orange, mTangerine, tdTomato) or any other suitable fluorescent protein. The fluorescent based reporter can be formed from two non-functional fragments (e.g., the C-terminus and the N-terminus) of a fluorescent protein (e.g., GFP, YFP, or RFP). These fragments can be referred to as a split-fluorescent protein (e.g., split-GFP, split-YFP, split-RFP). Accordingly, the fusion protein can comprise a fragment of a split-GFP, a split-YFP, a split-RFP.

In further embodiments, the fluorescent based reporter can be formed from a dimerization dependent system. Dimerization-dependent fluorophores are advantageously reversible with higher brightness from complemented sensor. In this system, a pair of a quenched fluorescent protein (e.g., ddGFPA, ddRFPA, ddYFPA) and a non-fluorogenic fluorescent protein (ddGFPB, ddRFPB, ddYFPB) form a heterodimer that can result in improved fluorescence. Accordingly, in various embodiments the fusion protein can comprise a monomer (e.g., ddGFPA, ddRFPA, ddYFPA, ddGFPB, ddRFPB, ddYFPB) of a dimerization dependent system.

In various embodiments, the complementary set of fragments or proteins can comprise a luminescent protein. Luminescent proteins, in contrast to fluorescent reporters which rely on photo-excitation, act upon a substrate to release a signal. In addition, split luciferase proteins can reversibly associate allowing for greater control in various assays. Luminescence is often preferable to fluorescence in cells or animals because it does not require excitation and therefore does not risk photobleaching or tissue damage. However, various luminescent proteins provide differing levels of signal and not all are easily split and recombined in a living system. Achieving usable signals from split luciferase is technically challenging because (1) it is difficult to express, (2) the luminescence of split luciferase is typically 100-1000 fold less than intact luciferase, and (3) the wavelength emitted by some luciferases exhibits poor penetration in tissues.

In various embodiments, the split luciferase can comprise firefly luciferase or a derivative thereof (e.g., AKA-Luc).

Although useful, the bulkiness of firefly luciferase can interfere with the function of domains fused to its fragments in complementation assays. Accordingly, in various embodiments, the fusion proteins can comprise a split luciferase protein other than firefly luciferase. Non-limiting examples include Renilla luciferase, Nanoluc luciferase and derivatives thereof. In various embodiments, the luminescent protein comprises nanoluciferase (NanoLuc). NanoLuc is a 19.1 kDa luciferase enzyme that uses the substrate furimazine to produce high intensity, glow-type luminescence. Its strong signal and stability provides advantages over other luciferases.

In further embodiments, the fusion protein can further comprise a second fluorescent protein that can be excited by light emitted by the fluorescent or luminescent protein (or heterodimer). This fluorescent or bioluminescent resonance energy transfer (FRET/BRET) can amplify the reporter signal and/or stabilize the linked fragment of the luminescent or fluorescent sensor. This has an added advantage of increasing tissue penetrance and is particularly useful when paired with one of the luminescent proteins described above. In various embodiments, the second fluorescent protein is mOrange (e.g., LSSmOrange), cpVenus, or GFP. In various embodiments, the second fluorescent protein is mOrange.

### 2. Poly-ADP Ribose Binding Domain.

The fusion proteins described herein further comprise a poly-ADP ribose (PAR) binding domain. Various PAR binding domains are known in the art. Exemplary types are macro domains, WWE domains and PBZ domains.

In various embodiments, the PAR binding domain comprises a macro domain. A “macro” domain bears a similarity to the C-terminal domain of a histone H2A variant called MacroH2A and can comprise about 130 to about 190 amino acids that adopt a distinct fold consisting of a central beta sheet surrounded by four to six helices. They can bind to free ADP-ribose or to the terminus of a growing poly-ADP chain and so can detect both poly and mono-PAR. Macro domains are found in many protein families, including glycohydrolases. In various embodiments, the PAR binding domain comprises a macro domain derived from a glycohydrolase (e.g., ADP ribose glycohydrolase AF1521). In various embodiments, the PAR binding domain comprises a macro domain derived from macroH2A, ALC1/CHD1L, or C6orf130/TARG.

In various embodiments, the PAR binding domain in the fusion protein can comprise at least 80%, at least 85%, at least 90%, at least 95%, at least 96%, at least 97%, at least 98%, at least 99% or 100% sequence identity to an illustrative macro domain from ADP ribose glycohydrolase AF1521 provided herein as SEQ ID NO: 1.

SEQ ID NO: 1:
MERRTLIMEVLFEAKVGDITLKLAQGDITQYPAKAIVNAANKRLEHGGG
VAYAIAKACAGDAGLYTEISKKAMREQFGRDYIDHGEVVVTPAMNLEER
GIKYVFHTVGPICSGMWSEELKEKLYKAFLGPLEKAEEMGVESIAFPAV
SAGIYGCDLEKVVETFLEAVKNFKGSAVKEVALVIYDRKSAEVALKVFE
RSL

In various embodiments, the PAR binding domain comprises a WWE domain. A “WWE domain” is an art-recognized moiety that typically binds to iso-ADP residues and accordingly, binds to poly-PAR. WWE domains, which are recognized by conserved W (tryptophan) and E (glutamate) residues, are found in many protein families, including E3 ubiquitin ligases In various embodiments, the PAR binding domain comprises a WWE domain derived from an E3 ubiquitin ligase (e.g., a RNF146/Iduna E3 ubiquitin ligase).

In various embodiments, the PAR binding domain in the fusion protein can comprise at least 80%, at least 85%, at least 90%, at least 95%, at least 96%, at least 97%, at least 98%, at least 99% or 100% sequence identity to an illustrative WWE domain from RNF 146 E3 ubiquitin ligase provided herein as SEQ ID NO: 2.

SEQ ID NO: 2:
MGNGEYAWYYEGRNGWWQYDERTSRELEDAFSKGKKNTEMLIAGFLYVA
DLENMVQYRRNEHGRRRKIKRDIIDIPKKGVAGLR

In various embodiments, the PAR binding domain includes a PAR-binding zinc finger (PBZ) domain. The binding to PAR by PBZ domains is thought to be structurally similar to the way macrodomains recognize PAR, and tandem repeats of PBZ domains enhance their ability to bind PAR. Recent studies have shown that the PBZ domains recognize branched forms of PAR chain, that is predominantly generated by PARP-2, this distinguishes PBZ from WWE or macrodomains that are capable of recognizing PAR generated by PARP-1 that can be linear. In various embodiments, the PBZ domain comprises a PBZ domain derived from aprataxin polynucleotide kinase (PNK)-like factor (APLF).

In various embodiments, the PAR binding domain in the fusion protein comprises at least 80%, at least 85%, at least 90%, at least 95%, at least 96%, at least 97%, at least 98%, at least 99% or 100% sequence identity to a PBZ domain derived from aprataxin polynucleotide kinase (PNK)-like factor (APLF) provided herein as SEQ ID NO: 3.

SEQ ID NO: 3:
MDSVLQGSEGNKVKRTSCMYGANCYRKNPVHFQHFSHPGDSDYGGVQIV
GQDETDDRPEC

In still further embodiments, the PAR binding domain does not comprise a PBZ domain (e.g., a PBZ domain derived from APLF). Accordingly, in various embodiments, the PAR binding domain in the fusion protein has less than 90%, less than 85%, less than 80%, less than 75%, less than 70%, less than 65%, or less than 60% sequence homology to SEQ ID NO: 3. In various embodiments, the PAR binding domain does not comprise a PBZ domain having an amino acid sequence comprising SEQ ID NO: 3.

In various embodiments, at least one of the PAR binding domains in the fusion protein pair comprises a WWE domain. In further embodiments, both of the two PAR binding domains in the fusion protein pair comprise the WWE domains. Alternatively, one fusion protein can comprise a macro domain and the other fusion protein can comprise a WWE domain.

### II. Methods of Constructing Fusion Proteins

Methods for preparing and expressing the fusion proteins in various systems are provided herein. Generally, fusion proteins can be expressed from an expression vector comprising a nucleic acid sequence that can encode for one or more of the fusion proteins.

Illustrative nucleic acids that can be used in the following methods to encode all or some of the fusion proteins are provided in the Sequence Listing filed herewith and described in Table 1, below. Use of these nucleic acids are described in more detail in the Examples described below. Table 1 provides four illustrative nucleic acid sequences (SEQ ID NOs: 4 to 7) which encode various PAR sensors comprising one of SEQ ID NOs: 11 to 15. These sensors all comprise a WWE domain (SEQ ID NO: 2) encoded by SEQ ID NO: 9. It would be clear to an ordinary person in the art that the portion of SEQ ID NO 4, 5, 6, or 7 comprising SEQ ID NO: 9 can be replaced with SEQ ID NO: 8 or 10 to enable expression of a PAR tracker comprising a macrodomain or a PBZ domain respectively. Likewise, SEQ ID NOs 4, 5, 6, and 7 each encode for either a dimerization dependent GFP protein or a split luminescent protein. It would be equally clear to one of ordinary skill in the art to replace the nucleotides encoding the reporter protein with a nucleic acid sequence encoding a different reporter.

TABLE 1
Illustrative nucleic acid sequences and encoded polypeptides according
to various aspects of the disclosure

| | SEQ | | SEQ |
|---|---|---|---|
| | ID | | ID |
| Nucleic Acid Description | NO: | Encoded Polypeptide | NO: |
| pINDUCER Flag-WWE-Linker- | 4 | Flag-AF-Linker-ddGFPA | 11 |
| ddGFPA-IRES-HA-WWE-Linker- | | HA-WWE-Linker-ddGFPB | 12 |
| ddGFPB-IRES-mCherry-NLS | | | |
| pET19b WWE-Linker-ddGFPA | 5 | His-WWE-Linker-ddGFPA | 13 |
| pET19b WWE-Linker-ddGFPB | 6 | His-WWE-Linker-ddGFPB | 14 |
| pINDUCER PAR-T NanoLuc | 7 | Flag-WWE-Linker-NanoLuc-N | 15 |
| LssmOrange | | Flag-WWE-Linker-NanoLuc-C- | 16 |
| | | LSSmOrange | |
| Macrodomain from AF 1521 | 8 | Macrodomain from AF1521 | 1 |
| WWE | 9 | WWE | 2 |
| PBZ1/2 | 10 | PBZ1/2 | 3 |

In various embodiments, any of fusion proteins disclosed herein can be produced via, e.g., conventional recombinant technology. In some examples, DNA encoding any of the fusion proteins disclosed herein can be readily isolated and sequenced using conventional procedures (e.g., by using oligonucleotide probes that are capable of binding specifically to genes encoding a polypeptide sequence). Once isolated, the DNA may be placed into one or more expression vectors, which are then transfected into host cells such as E. coli cells, simian COS cells, Chinese hamster ovary (CHO) cells, Human Embryotic Kidney (HEK) 293 cells or myeloma cells that do not otherwise produce the fusion proteins disclosed herein. In additional embodiments, the expression vectors can be transfected into a host cell having an altered level of PARP activity (i.e., a cancer cell). In further embodiments, the expression vectors can be delivered to a tissue or a mammal using a viral vector or other standard means. The DNA can then be modified accordingly for generating any of the compositions disclosed herein.

In some examples, any of the fusion proteins disclosed herein can be prepared by recombinant technology as exemplified below.

Nucleic acids encoding any of fusion proteins disclosed herein can be cloned into one expression vector, each nucleotide sequence being in operable linkage to a suitable promoter. In some examples, each of the nucleotide sequences encoding any of fusion proteins disclosed herein can be in operable linkage to a distinct prompter. Alternatively, the nucleotide sequences encoding any of the fusion proteins disclosed herein can be in operable linkage with a single promoter, such that one or more proteins are expressed from the same promoter. When necessary, an internal ribosomal entry site (IRES) can be inserted between protein encoding sequences.

In some examples, the nucleotide sequences encoding any of fusion proteins disclosed herein can be cloned into two vectors, which can be introduced into the same or different cells. When any of the fusion proteins disclosed herein are expressed in different cells, each of them can be isolated from the host cells expressing such and the isolated proteins can be mixed and incubated under suitable conditions allowing, for example, methods of detecting PAR levels as disclosed herein.

Generally, a nucleic acid sequence encoding one or all of fusion proteins disclosed herein can be cloned into a suitable expression vector in operable linkage with a suitable promoter using methods known in the art. For example, the nucleotide sequence and vector can be contacted, under suitable conditions, with a restriction enzyme to create complementary ends on each molecule that can pair with each other and be joined together with a ligase. Alternatively, synthetic nucleic acid linkers can be ligated to the termini of a gene. These synthetic linkers contain nucleic acid sequences that correspond to a particular restriction site in the vector. The selection of expression vectors/promoter would depend on the type of host cells for use in producing the decoy fusion proteins.

A variety of promoters can be used for expression of any of the fusion proteins disclosed herein, including, but not limited to, cytomegalovirus (CMV) intermediate early promoter, a viral LTR such as the Rous sarcoma virus LTR, HIV-LTR, HTLV-1 LTR, the simian virus 40 (SV40) early promoter, E. coli lac UV5 promoter, and the herpes simplex tk virus promoter.

Regulatable promoters can also be used. Such regulatable promoters can include those using the lac repressor from E. coli as a transcription modulator to regulate transcription from lac operator-bearing mammalian cell promoters [Brown, M. et al., Cell, 49:603-612 (1987)], those using the tetracycline repressor (tetR) [Gossen, M., and Bujard, H., Proc. Natl. Acad. Sci. USA 89:5547-5551 (1992); Yao, F. et al., Human Gene Therapy, 9:1939-1950 (1998); Shockelt, P., et al., Proc. Natl. Acad. Sci. USA, 92:6522-6526 (1995)]. Other systems include FK506 dimer, VP16 or p65 using astradiol, RU486, diphenol murislerone, or rapamycin.

Regulatable promoters that include a repressor with the operon can be used. In one embodiment, the lac repressor from E. coli can function as a transcriptional modulator to regulate transcription from lac operator-bearing mammalian cell promoters [M. Brown et al., Cell, 49:603-612 (1987); Gossen and Bujard (1992); M. Gossen et al., Natl. Acad. Sci. USA, 89:5547-5551 (1992)] combined the tetracycline repressor (tetR) with the transcription activator (VP 16) to create a tetR-mammalian cell transcription activator fusion protein, tTa (tetR-VP 16), with the tetO-bearing minimal promoter derived from the human cytomegalovirus (hCMV) major immediate-early promoter to create a tetR-tet operator system to control gene expression in mammalian cells. In one embodiment, a tetracycline inducible switch is used. The tetracycline repressor (tetR) alone, rather than the tetR-mammalian cell transcription factor fusion derivatives can function as potent trans-modulator to regulate gene expression in mammalian cells when the tetracycline operator is properly positioned downstream for the TATA element of the CMVIE promoter (Yao et al., Human Gene Therapy, 10(16): 1392-1399 (2003)). One particular advantage of this tetracycline inducible switch is that it does not require the use of a tetracycline repressor-mammalian cells transactivator or repressor fusion protein, which in some instances can be toxic to cells (Gossen et al., Natl. Acad. Sci. USA, 89:5547-5551 (1992); Shockett et al., Proc. Natl. Acad. Sci. USA, 92:6522-6526 (1995)), to achieve its regulatable effects.

Additionally, vectors used herein can contain, for example, some or all of the following: a selectable marker gene, such as the neomycin gene for selection of stable or transient transfectants in mammalian cells; enhancer/promoter sequences from the immediate early gene of human CMV for high levels of transcription; transcription termination and RNA processing signals from SV40 for mRNA stability; SV40 polyoma origins of replication and ColE1 for proper episomal replication; internal ribosome binding sites (IRESes), versatile multiple cloning sites; and T7 and SP6 RNA promoters for in vitro transcription of sense and antisense RNA. Suitable vectors and methods for producing vectors containing transgenes are well known and available in the art.

One or more vectors (e.g., expression vectors) comprising nucleic acids encoding any of the fusion proteins disclosed herein may be introduced into suitable host cells for producing the any of fusion proteins. The host cells can be cultured under suitable conditions for expression of any of fusion proteins disclosed herein. Such fusion proteins can be recovered by the cultured cells (e.g., from the cells or the culture supernatant) via a conventional method, e.g., affinity purification. If necessary, any of the host cells disclosed herein can be incubated under suitable conditions for a suitable period of time allowing for production of the fusion proteins.

In some embodiments, methods for preparing any of the fusion proteins disclosed herein described herein can involve a recombinant expression vector that encodes all components of the any of the fusion proteins also disclosed herein. The recombinant expression vector can be introduced into a suitable host cell (e.g., a HEK293T cell or a dhfr-CHO cell) by a conventional method, e.g., calcium phosphate-mediated transfection. Positive transformant host cells can be selected and cultured under suitable conditions allowing for the expression of any of the fusion proteins disclosed herein which can be recovered from the cells or from the culture medium. When necessary, any of the fusion proteins recovered from the host cells can be incubated under suitable conditions allowing for the formation of decoy fusion protein homodimers.

As described further below, the fusion proteins can be expressed in vivo (for example, in a mammal). Accordingly, in various embodiments expression vectors encoding for any of the fusion proteins described herein can be formulated in a viral vector (e.g., an adenoviral vector), a nanoparticle or other delivery module to facilitate delivery into a target organ, tissue or cell in an animal. In other embodiments, the fusion proteins can be expressed in vivo by xenograft transplantation of transfected cells into the animal. Suitable cells that can be transfected prior to xenograft experiments are known in the art. As an example, MDA-MB-231-luc cells can be used. As noted above, vectors for expressing fusion proteins in transplanted cell lines can be under the control of an inducible promoter (like doxycycline) to allow for controlled expression once the xenograft has been established.

### III. Methods of Use

In various embodiments, the fusion proteins and systems provided herein can be used to track PAR levels in a cell or a tissue. Accordingly, in various embodiments, a method of detecting poly-ADP ribose polymerase (PARP) activity in a cell or tissue suspected of having PARP activity is provided. The method comprises (a) introducing the first and second fusion proteins as disclosed herein into the cell or tissue; (b) maintaining the cell or tissue for a time and under conditions sufficient for the first and second fusion proteins to bind to one or more poly-ADP ribose (PAR) chains and combine to produce a signal; and (c) detecting the signal, wherein the signal is proportional to the PARP activity in the system.

In various embodiments, introducing the first and second fusion protein comprises introducing one or more expression vectors described above comprising one or more nucleic acid sequences encoding the first and second fusion proteins, and maintaining the cell or tissue for a time and under conditions sufficient for the cell or cells in the tissue to express the first and second fusion proteins.

As described above, the first and second fusion proteins in these methods can comprise complementary fragments of a reporter protein that are capable of combining to produce a signal. In various embodiments, the complementary fragments comprise fragments of a split fluorescent protein (e.g., a split-GFP, a split-YFP, a split-RFP). In further embodiments, the complementary fragments comprise fragments of a split luminescent protein (e.g., luciferase). In further embodiments, the first and second fusion proteins in these methods can comprise monomers of a dimerization dependent reporter system that are capable of combining to form a heterodimer that produces a signal.

In various embodiments, the reporter protein (and fragments thereof) requires a substrate to generate a signal. For example, the substrate can comprise furimazine. Accordingly, the method can further comprise introducing a substrate (like furimazine) that can be acted upon by the reporter protein to the cell or tissue.

In various embodiments, cell or tissue is in vitro, in situ, or in vivo. In further embodiments, the cell or tissue lacks cell lysate. An advantage of the system described herein is its ability to work in living cells or tissues and does not require a purely ‘in vitro’ method. Accordingly, in various embodiments, the cells or tissue comprise a living cell or living tissue. In various embodiments, the cells or tissues are in a living animal.

In various embodiments, the PAR trackers described herein can be used to detect PARP levels and activity in a variety of systems. For example, DNA damage is a hallmark of cancer which usually leads to elevated PARP activity. Accordingly, the methods can further comprise detecting PARP levels in a cancerous system (i.e., using cancer cells in vitro or in an animal model). Given the role PARP has in repairing DNA damage, various PARP inhibitors or activators are being tested as cancer therapeutics. Accordingly, in various embodiments these potential therapeutics (PARP inhibitors or activators) can be tested in a system (in vitro or in vivo) using the PAR trackers herein. Because these trackers can be expressed in living tissue they allow for experiments where an agent's effect can be tracked in real time. This provides an advantage over current methods where a tissue or cell must be lysed prior to PARP analysis.

Accordingly, in some embodiments, disclosed is a method of identifying a potential therapeutic comprising (a) introducing the first and second fusion proteins described herein into a cell, tissue or animal, (b) applying the potential therapeutic to the cell, tissue or animal, (c) maintaining the cell or tissue or animal for a time and under conditions sufficient for the first and second fusion proteins to bind to one or more poly-ADP ribose (PAR) chains and combine to produce a signal; and (c) detecting the signal, wherein the signal is indicative of the efficacy of the potential therapeutic. In various embodiments, the potential therapeutic comprises a PARP inhibitor, a poly (ADP ribose) glycohydrolase (PARG) inhibitor, or another agent that is suspected to modulate the activity of PARP in a system.

In further embodiments, disclosed is a method of assessing the effectiveness of a potential therapeutic to treat cancer comprising: (a) introducing the first and second fusion proteins described herein into a cancerous cell, a cancerous tissue or an animal having cancer, (b) applying the potential therapeutic to the cancerous cell, cancerous tissue or cancerous animal (c) maintaining the cancerous cell, cancerous tissue or cancerous animal for a time and under conditions sufficient for the first and second fusion proteins to bind to one or more poly-ADP ribose (PAR) chains and combine to produce a signal; and (c) detecting the signal, wherein the signal is indicative of the efficacy of the potential therapeutic to treat cancer. In various embodiments, the potential therapeutic comprises a PARP inhibitor, a poly (ADP ribose) glycohydrolase (PARG) inhibitor, or another agent that is suspected to modulate the activity of PARP in a cancerous system.

### IV. Kits

The present disclosure provides kits for performing any of the methods disclosed herein. In certain embodiments, kits herein can be used to prepare at least one of the compositions disclosed herein. In some examples, kits herein can be used to generate one or more of the fusion proteins disclosed herein. In some examples, kits herein can be used to generate any of the constructs disclosed herein. In some examples, kits herein can contain any of the materials needed to generate recombinant constructs disclosed herein, wherein the materials can be any of those known to the skilled artisan to be useful in standard molecular biology protocols such as, but not limited to, expression vectors, restriction enzymes, PCR buffers and enzymes, resins, and the like. In some embodiments, kits herein can further include instructions on how to generate any of the compositions disclosed herein (e.g., fusion proteins, expression vectors, etc.).

In certain embodiments, kits herein can be used to perform methods of detecting poly-ADP ribose polymerase activity in a cell or tissue as disclosed herein. In some examples, kits can have components needed to generate any of the compositions disclosed herein (e.g., fusion proteins, expression vectors, etc.) used in the methods described above. In some examples, kits can have pre-paired compositions, at least one pre-paired component of a composition, or a combination thereof. In some embodiments, kits herein can have a first or second fusion protein as described herein, or an expression vector able to express the first or second fusion protein in a cell, tissue or animal. In some embodiments, kits herein can have instructions on how to perform any of the methods of detecting PARP activity, or testing potential therapeutics as disclosed herein.

In certain embodiments, kits herein can include at least one container and/or a label or package insert(s) on or associated with the container. In some embodiments, the invention provides articles of manufacture comprising contents of the kits described above. The kits of this invention can be in suitable packaging. Suitable packaging includes, but is not limited to, vials, bottles, jars, flexible packaging (e.g., sealed Mylar or plastic bags), and the like. Any instructions included in kits herein can be written instructions on a label or package insert (e.g., a paper sheet included in the kit), but machine-readable instructions (e.g., instructions carried on a magnetic or optical storage disk) are also acceptable.

### IV. Terminology

Definitions and methods described herein are provided to better define the present disclosure and to guide those of ordinary skill in the art in the practice of the present disclosure. Unless otherwise noted, terms are to be understood according to conventional usage by those of ordinary skill in the relevant art.

In some embodiments, numbers expressing quantities of ingredients, properties such as molecular weight, reaction conditions, and so forth, used to describe and claim certain embodiments of the present disclosure are to be understood as being modified in some instances by the term “about.” In some embodiments, the term “about” is used to indicate that a value includes the standard deviation of the mean for the device or method being employed to determine the value. In some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the present disclosure are approximations, the numerical values set forth in the specific examples are reported as precisely as practicable. The numerical values presented in some embodiments of the present disclosure may contain certain errors necessarily resulting from the standard deviation found in their respective testing measurements. The recitation of ranges of values herein is merely intended to serve as a shorthand method of referring individually to each separate value falling within the range. Unless otherwise indicated herein, each individual value is incorporated into the specification as if it were individually recited herein.

In some embodiments, the terms “a” and “an” and “the” and similar references used in the context of describing a particular embodiment (especially in the context of certain of the following claims) can be construed to cover both the singular and the plural, unless specifically noted otherwise. In some embodiments, the term “or” as used herein, including the claims, is used to mean “and/or” unless explicitly indicated to refer to alternatives only or the alternatives are mutually exclusive.

The terms “comprise,” “have” and “include” are open-ended linking verbs. Any forms or tenses of one or more of these verbs, such as “comprises,” “comprising,” “has,” “having,” “includes” and “including,” are also open-ended.

For example, any method that “comprises,” “has” or “includes” one or more steps is not limited to possessing only those one or more steps and can also cover other unlisted steps. Similarly, any composition or device that “comprises,” “has” or “includes” one or more features is not limited to possessing only those one or more features and can cover other unlisted features.

All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the present disclosure and does not pose a limitation on the scope of the present disclosure otherwise claimed. No language in the specification should be construed as indicating any non-claimed element essential to the practice of the present disclosure.

Groupings of alternative elements or embodiments of the present disclosure disclosed herein are not to be construed as limitations. Each group member can be referred to and claimed individually or in any combination with other members of the group or other elements found herein. One or more members of a group can be included in, or deleted from, a group for reasons of convenience or patentability. When any such inclusion or deletion occurs, the specification is herein deemed to contain the group as modified thus fulfilling the written description of all Markush groups used in the appended claims.

All publications, patents, patent applications, and other references cited in this application are incorporated herein by reference in their entirety for all purposes to the same extent as if each individual publication, patent, patent application or other reference was specifically and individually indicated to be incorporated by reference in its entirety for all purposes. Citation of a reference herein shall not be construed as an admission that such reference is prior art to the present disclosure.

Having described the present disclosure in detail, it will be apparent that modifications, variations, and equivalent embodiments are possible without departing the scope of the present disclosure defined in the appended claims. Furthermore, it should be appreciated that all examples in the present disclosure are provided as non-limiting examples.

### Examples

The following examples are included to demonstrate preferred embodiments of the disclosure. It should be appreciated by those of skill in the art that the techniques disclosed in the examples that follow represent techniques discovered by the inventor to function well in the practice of the present disclosure, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the present disclosure.

### Materials and Methods

### Cell Culture and Treatments

HeLa, 293T, and MCF7 cells were obtained from the American Type Cell Culture, and MDA-MB-231-luc cells were obtained from Dr. Srinivas Malladi. They were cultured in DMEM (Sigma-Aldrich, D5796) supplemented with 10% fetal bovine serum (Sigma, F8067) and 1% penicillin/streptomycin. The cells were regularly verified as mycoplasma-free.

Cells were treated with various inhibitors as described herein. For inhibition of nuclear PARPs, cells were treated with PJ-34 (20 M; Enzo, ALX-270) or Olaparib (20 UM; MedChem Express, HY-10162) for 2 hours. For inhibition of PARG, cells were treated with PDD 00017273 (20 UM; MedChem Express, HY-108360) for 2 hours. For UV-induced DNA damage, the cells were harvested 15 minutes after treatment with 50 mJ/cm2 UV irradiation.

### Vectors for Ectopic Expression and Knockdown

The vectors described below were generated using the oligonucleotide primers described in the next section. All constructs were verified by sequencing.

Mammalian expression vectors. The plasmid for Dox-inducible expression of the ddGFP PAR-T constructs were generated using a cDNA for ddGFP-A (Addgene, 40286) or ddGFP-B (Addgene, 40287), cDNA for the PAR binding domains was amplified from the pET19b constructs. The cDNAs were assembled and cloned first into pCDNA3 vector and then into pInducer20 or pET19b vectors using Gibson assembly (NEB, E2621). The split luciferase constructs were synthesized as gene blocks (Integrated DNA technologies), and then cloned into pInducer20 vectors using Gibson assembly. The sequences of the various nucleic acid constructs and encoded polypeptides as used in these experiments are provided in the attached Sequence Listing and summarized in the Table below (reproduced from Table 1 above).

TABLE
Nucleic Acid Constructs and Encoded Polypeptides

| | SEQ | | SEQ |
|---|---|---|---|
| | ID | | ID |
| Nucleic Acid Description | NO: | Encoded Polypeptide | NO: |
| pINDUCER Flag-WWE-Linker- | 4 | Flag-AF-Linker-ddGFPA | 11 |
| ddGFPA-IRES-HA-WWE-Linker- | | HA-WWE-Linker-ddGFPB | 12 |
| ddGFPB-IRES-mCherry-NLS | | | |
| pET19b WWE-Linker-ddGFPA | 5 | His-WWE-Linker-ddGFPA | 13 |
| pET19b WWE-Linker-ddGFPB | 6 | His-WWE-Linker-ddGFPB | 14 |
| pINDUCER PAR-T NanoLuc | 7 | Flag-WWE-Linker-NanoLuc-N | 15 |
| LssmOrange | | Flag-WWE-Linker-NanoLuc-C- | 16 |
| | | LSSmOrange | |
| Macrodomain from AF1521 | 8 | Macrodomain from AF1521 | 1 |
| WWE | 9 | WWE | 2 |
| PBZ1/2 | 10 | PBZ1/2 | 3 |

### Primers

TABLE 2
Primers for cloning ddGFPA-ddGFPB into pCDNA3

| | | SEQ ID |
|---|---|---|
| Name | Sequence (5′-3′) | NO: |
| Forward 1 | AGGGGCGGAATTCCTCTAGTTCAATGCCCCAGGTGGTG | 17 |
| Reverse 1 | AGGGGCGGAATTCCTCTAGTTCAATGCCCCAGGTGGTG | 18 |
| Forward 2 | ATTACGCTCTTGAAGCAACCATGGCCACCATCAAAGAGTTCATGC | 19 |
| Reverse 2 | TAGGGCCCTCTAGATGCATGTTACTTGTACCGCTCGTC | 20 |

TABLE 3
Primers for cloning WWE-ddGFPA and WWE-ddGFPB into pCDNA3

| | | SEQ |
|---|---|---|
| | | ID |
| Name | Sequence (5′-3′) | NO: |
| Forward 1 | ATGACAAGCTTGAAGCAACCGGAAATGGTGAATATGCATGGTATTATG | 21 |
| Reverse 1 | AGGGGCGGAATTCCTCTAGTTCAATGCCCCAGGTGGTG | 22 |
| Forward 2 | ATTACGCTCTTGAAGCAACCGGAAATGGTGAATATGCATG | 23 |
| Reverse 2 | TAGGGCCCTCTAGATGCATGTTACTTGTACCGCTCGTC | 24 |

TABLE 4
Primers for cloning ddGFPA or ddGFPB into pET19b

| | | SEQ ID |
|---|---|---|
| Name | Sequence (5′-3′) | NO: |
| pET19b- | TATCGACGACGACGACAAGCATATGCTCGAGATGGCGAGCAAGA | 25 |
| ddGFPA | GCGAG | |
| Forward | | |
| pET19b- | TCGGGCTTTGTTAGCAGCCGGATCCTCAATGCCCCAGGTGGTG | 26 |
| ddGFPA | | |
| Reverse | | |
| pET19b- | TATCGACGACGACGACAAGCATATGCTCGAGACCATCAAAGAGTT | 27 |
| ddGFPB | CATGC | |
| Forward | | |
| pET19b- | TCGGGCTTTGTTAGCAGCCGGATCCTTACTTGTACCGCTCGTC | 28 |
| ddGFPB | | |
| Reverse | | |

TABLE 5
Primers for cloning WWE-ddGFPA or WWE-ddGFPB into pET19b

| | | SEQ |
|---|---|---|
| | | ID |
| Name | Sequence (5′-3′) | NO: |
| pET19b-WWE- | TATCGACGACGACGACAAGCATATGCTCGAGGGAAATGGTG | 29 |
| ddGFPA Forward: | AATATGCATG | |
| pET19b-WWE- | TCGGGCTTTGTTAGCAGCCGGATCCTCAATGCCCCAGGTG | 30 |
| ddGFPA Reverse: | GTG | |
| pET19b-WWE- | TATCGACGACGACGACAAGCATATGCTCGAGGGAAATGGTG | 31 |
| ddGFPB Forward: | AATATGCATG | |
| pET19b-WWE- | TCGGGCTTTGTTAGCAGCCGGATCCTTACTTGTACCGCTCG | 32 |
| ddGFPB Reverse: | TC | |

TABLE 6
Primers for cloning MacroH2A.1-ddGFPA or MacroH2A.1-ddGFPB into

| | | SEQ |
|---|---|---|
| | | ID |
| Name | Sequence | NO: |
| Forward 1 | TATCGACGACGACGACAAGCATATGCTCGAGGGTGAAG | 33 |
| | TCAGTAAGGCAGC | |
| Reverse 1 | AGAATTCTAGGTTGGCGTCCAGCTTGGC | 34 |
| pET19b-MacroH2A.1- | GGACGCCAACCTAGAATTCTCGACAGGGCATG | 35 |
| ddGFPA Forward: | | |
| pET19b-MacroH2A.1- | TCGGGCTTTGTTAGCAGCCGGATCCTCAATGCCCCAGG | 36 |
| ddGFPA Reverse: | TGGTG | |
| pET19b-MacroH2A.1- | GGACGCCAACCTAGAATTCTCGACAGGG | 37 |
| ddGFPB Forward: | | |
| pET19b-MacroH2A.1- | TCGGGCTTTGTTAGCAGCCGGATCCTTACTTGTACCGCT | 38 |
| ddGFPB Reverse: | CGTC | |

TABLE 7
Primers for cloning PBZ-ddGFPA or PBZ-ddGFPB into pET19b

| | | SEQ |
|---|---|---|
| | | ID |
| Name | Sequence (5′-3′) | NO: |
| Forward 1: | TATCGACGACGACGACAAGCATATGCTCGAGGATTCAGTTC | 39 |
| | TACAAGGTTC | |
| Reverse 1: | AGAATTCTAGTGGAAGCGTATTATGTCTATATTC | 40 |
| pET19b-PBZ- | TACGCTTCCACTAGAATTCTCGACAGGGCATG | 41 |
| ddGFPA Forward: | | |
| pET19b-PBZ- | TCGGGCTTTGTTAGCAGCCGGATCCTCAATGCCCCAGGTG | 42 |
| ddGFPA Reverse: | GTG | |
| pET19b-PBZ- | TACGCTTCCACTAGAATTCTCGACAGGG | 43 |
| ddGFPB Forward: | | |
| pET19b-PBZ- | TCGGGCTTTGTTAGCAGCCGGATCCTTACTTGTACCGCTCG | 44 |
| ddGFPB Reverse: | TC | |

TABLE 8
Primers for cloning MacroAF-ddGFPA or PBZ-ddGFPB into pET19b

| | | SEQ |
|---|---|---|
| | | ID |
| Name | Sequence (5′-3′) | NO: |
| Forward 1 | TATCGACGACGACGACAAGCATATGCTCGAGATGGAACGG | 45 |
| | CGTACTTTAATC | |
| Reverse 1 | AGAATTCTAGAAGACTCCTCTCAAAGAC | 46 |
| pET19b-MacroAF- | GAGGAGTCTTCTAGAATTCTCGACAGGGCATG | 47 |
| ddGFPA Forward: | | |
| pET19b-PBZ- | TCGGGCTTTGTTAGCAGCCGGATCCTCAATGCCCCAGGTG | 48 |
| ddGFPA Reverse: | GTG | |
| pET19b-MacroAF- | GAGGAGTCTTCTAGAATTCTCGACAGGG | 49 |
| ddGFPB Forward | | |
| pET19b-PBZ- | TCGGGCTTTGTTAGCAGCCGGATCCTTACTTGTACCGCTCG | 50 |
| ddGFPB Reverse | TC | |

TABLE 9
Primers for cloning WWE-ddGFP sensors and control ddGFP into plnducer20

| | | SEQ ID |
|---|---|---|
| Name | Sequence (5′-3′) | NO: |
| Forward | TCCGCGGCCCCGAACTAGTGGCCACCATGGACTACAAG | 51 |
| Reverse | AGAGGGGCGGAATTCCTCTAGTCTTACTTGTACCGCTCGTC | 52 |

TABLE 10
Primers for cloning AF-ddGFP sensors into plnducer20

| | | SEQ ID |
|---|---|---|
| Name | Sequence (5′-3′) | NO: |
| Forward 1 | TCCGCGGCCCCGAACTAGTGGCCACCATGGACTACAAGGATGAC | 53 |
| | GATGACAAGCTTGAAGCAACCATGGAACGGCGTACTTTAATCATG | |
| Reverse 1 | TTCCTCTAGTTCAATGCCCCAGGTGGTG | 54 |
| Forward 2 | GGGGCATTGAACTAGAGGAATTCCGCCC | 55 |
| Reverse 2 | AGAGGGGCGGAATTCCTCTAGTCTTACTTGTACCGCTCGTC | 56 |

TABLE 11
Primers for cloning split firefly luciferase sensors into pCDNA3

| | | SEQ ID |
|---|---|---|
| Name | Sequence (5′-3′) | NO: |
| pCDNA3-WWE/MacroAF-LucN: | CAAGCTTGGTACCGAGCTCGGCCACCATG | 57 |
| Forward 1: | GACTACAAG | |
| pCDNA3-WWE/MacroAF-LucN: | CCATGGATCCTGAACTACCGGTCGATTC | 58 |
| Reverse 1 | | |
| pCDNA3-WWE/MacroAF-LucN: | CGGTAGTTCAGGATCCATGGAAGACGCC | 59 |
| Forward 2 | | |
| pCDNA3-WWE/MacroAF-LucN: | AGGGCCCTCTAGATGCATGCTCACATAATC | 60 |
| Reverse-2: | ATAGGTCCTCTGAC | |
| pCDNA3-WWE/MacroAF-LucC: | CAAGCTTGGTACCGAGCTCGGCCACCATG | 61 |
| Forward 1: | GACTACAAG | |
| pCDNA3-WWE/MacroAF-LucC: | GTCCGGATCCTGAACTACCGGTCGATTC | 62 |
| Reverse 1 | | |
| pCDNA3-WWE/MacroAF-LucC: | CGGTAGTTCAGGATCCGGACCTATGATTAT | 63 |
| Forward 2: | G | |
| pCDNA3-WWE/MacroAF-LucC: | AGGGCCCTCTAGATGCATGCTTACAATTTG | 64 |
| Reverse-2 | GACTTTCCG | |

TABLE 12
Primers for cloning split Nano luciferase sensors into plnducer20

| | | SEQ |
|---|---|---|
| | | ID |
| Name | Sequence (5′-3′) | NO: |
| Forward 1 | TCCGCGGCCCCGAACTAGTGATGGACTACAAGGATGAC | 65 |
| Reverse 1 | CTCCGCTTCCACTGTTGATGGTTACTCG | 66 |
| Forward 2 | CATCAACAGTGGAAGCGGAGCCACGAAC | 67 |
| Reverse-2 | GTTTAATTAATCATTACTACTTACTTGTACAGCTCGTCCATGC | 68 |

Knockdown of PARP1 and PARP2 Using siRNAs.

Commercially available siRNA oligos targeting PARP1 (Sigma, SASI_Hs01_0033277), PARP2 (Sigma, SASI_Hs01_0013-1488) and control siRNA (Sigma, SIC001) were transfected at a final concentration of 30 nM using Lipofectamine RNAiMAX reagent (Invitrogen, 13778150) according to the manufacturer's instructions. All experiments were performed 48 hours after siRNA transfection.

### Generation of Stable Cell Lines

Cells were transfected with lentiviruses for stable ectopic expression. Lentiviruses were generated by transfection of the pInducer20 constructs described above, together with an expression vector for the VSV-G envelope protein (pCMV-VSV-G, Addgene plasmid no. 8454), an expression vector for GAG-Pol-Rev (psPAX2, Addgene plasmid no. 12260), and a vector to aid with translation initiation (pAdVAntage, Promega) into 293T cells using GeneJuice transfection reagent (Novagen, 70967) according to the manufacturer's protocol. The resulting viruses were used to infect HeLa, MCF7 or MDA-MB-231 cells in the presence of 7.5 μg/mL polybrene 24 hours and 48 hours, respectively, after initial 293T transfection. Stably transduced cells were selected with 500 μg/mL G418 sulfate (Sigma, A1720). For inducible expression of RPL24, the cells were treated with 1 μg/mL Doxycycline for 24 hours.

### Preparation of Cell Lysates

Cells were cultured and treated as described above before the preparation of cell extracts. At the conclusion of the treatments, the cells were washed twice with ice-cold PBS and lysed with Lysis Buffer (20 mM Tris-HCl PH 7.5, 150 mM NaCl, 1 mM EDTA, 1 mM EGTA, 1% NP-40, 1% sodium deoxycholate, 0.1% SDS) containing 1 mM DTT, 250 nM ADP-HPD (Sigma, A0627), 10 UM PJ34 (Enzo, ALX-270), and 1× complete protease inhibitor cocktail (Roche, 11697498001). The cells were incubated in the Lysis Buffer for 30 minutes on ice and then centrifuged at full speed for 15 minutes at 4° C. in a microcentrifuge to remove the cell debris.

### Western Blotting

Protein concentrations of the cell lysates were determined using a Bio-Rad Protein Assay Dye Reagent (Bio-Rad, 5000006). Equal volumes of lysates containing the same concentrations of protein were boiled at 100° C. for 5 minutes after addition of ¼ volume of 4×SDS-PAGE Loading Solution (250 mM Tris, pH 6.8, 40% glycerol, 0.04% Bromophenol Blue, 4% SDS), run on 6% polyacrylamide-SDS gels, and transferred to nitrocellulose membranes. After blocking with 5% nonfat milk in TBST, the membranes were incubated with the primary antibodies described above in 1% non-fat milk in TBST with 0.02% sodium azide, followed by anti-rabbit HRP-conjugated IgG (1:5000) or anti-mouse HRP-conjugated IgG (1:5000). Immunoblot signals were detected using an ECL detection reagent (Thermo Fisher Scientific, 34577, 34095).

### Antibodies

The custom rabbit polyclonal antiserum against PARP-1 was generated in-house by using purified recombinant amino-terminal half of PARP-1 as an antigen (now available Active Motif, cat. no. 39559). The custom recombinant antibody-like anti-poly-ADP-ribose binding reagent (anti-PAR) was generated and purified in-house (now available from EMD Millipore, MABE1031). The other antibodies used were as follows: PARP-2 (Santa Cruz, sc-150X), α-Tubulin (Abcam, ab6046), goat anti-rabbit HRP-conjugated IgG (Pierce, 31460).

### Example 1: Using Dimerization-Dependent GFP-Based Reagents to Detect PARylation in Vitro

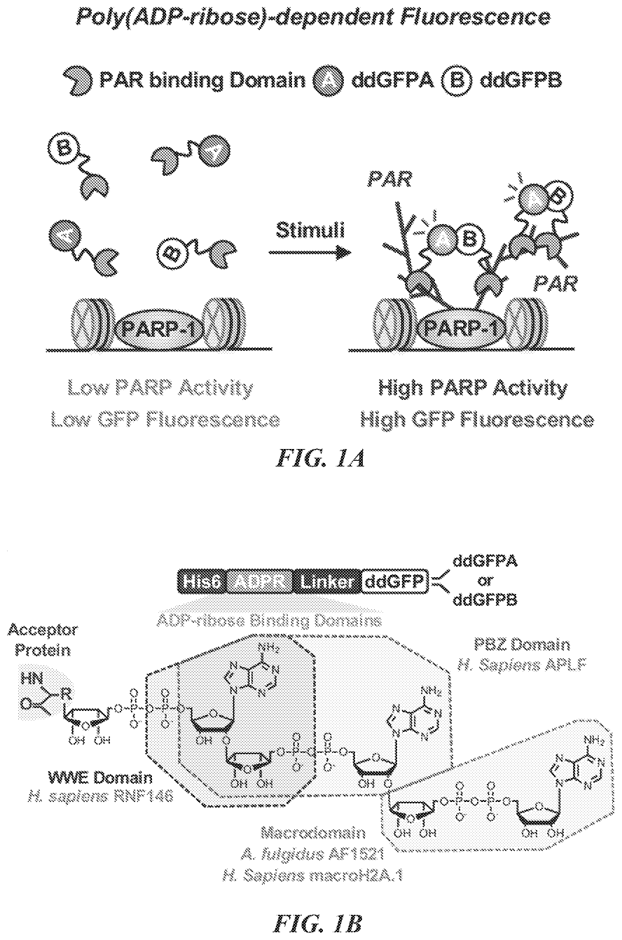

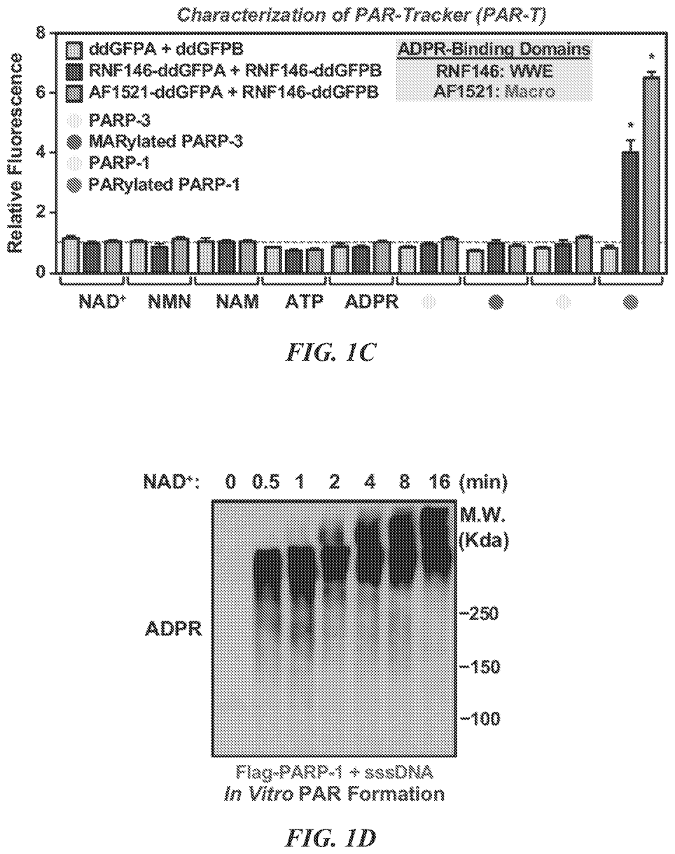

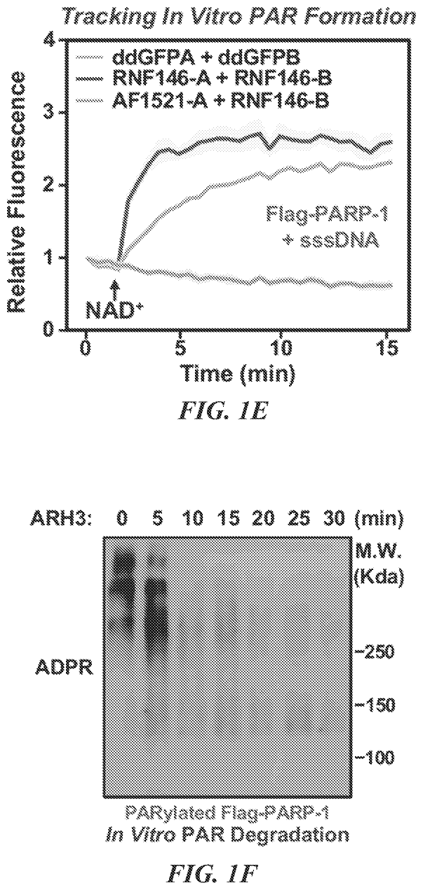

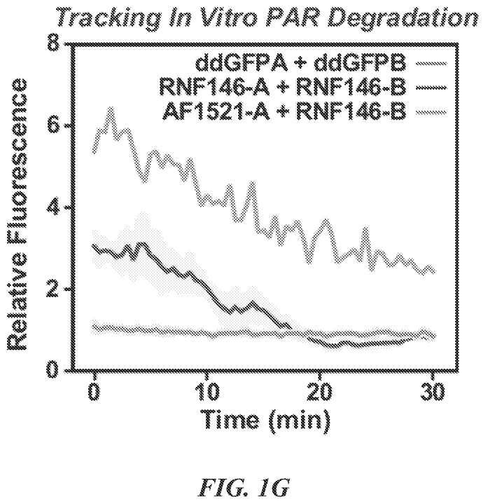

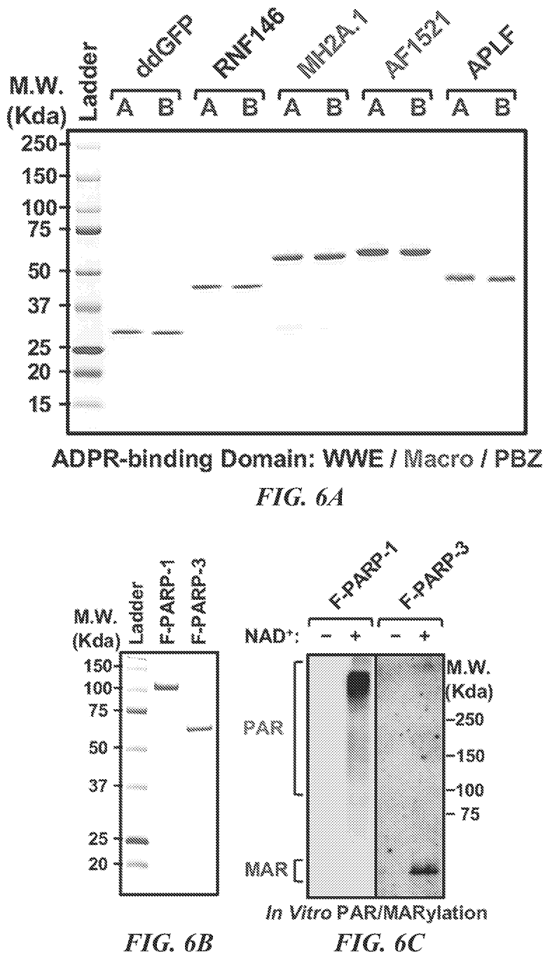

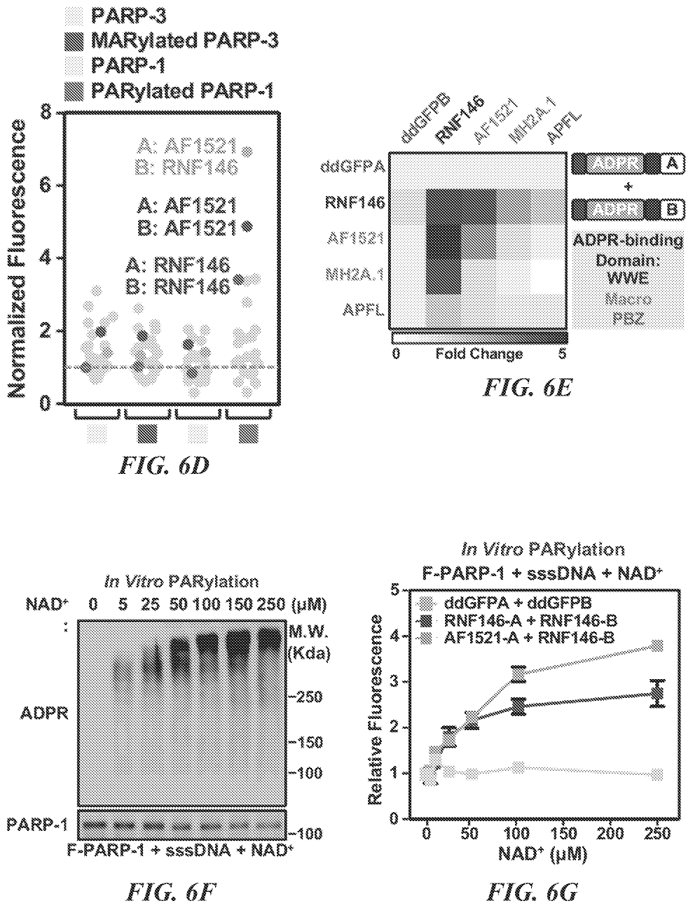

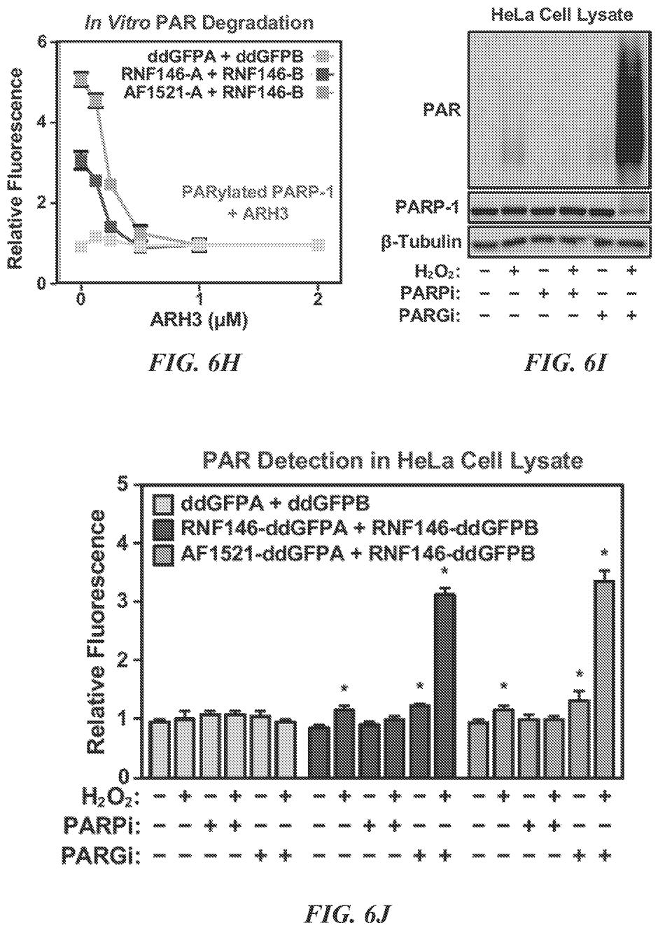

Dimerization-dependent GFP is a genetically encoded sensor that was initially developed to study protein interactions. In this system, a pair of a quenched GFP (ddGFPA) and a non-fluorogenic GFP (ddGFPB) form a heterodimer that can result in improved fluorescence. In this example, a ddGFP-based fluorescence sensor (fluorescent PAR-Trackers or PAR-T) was designed to allow for live cell imaging, with high signal-noise ratio. To achieve this, the various ADPR reader domains (ARBDs) were fused to ddGFP-A/B and purified as recombinant proteins (FIG. 1A, FIG. 1B, FIG. 6A). In vitro ADP-ribosylation assays were performed using recombinant PARP-1 (to detect PAR) and PARP-3 (to detect MAR) specifically (FIG. 6B and FIG. 6C). It was observed that of all the ARBD-ddGFP pairs tested, the WWE domain from RNF146, macrodomain from AF1521 and a combination of these performed well in recognizing PARylated PARP-1 specifically (FIG. 6D and FIG. 6E). These reagents recognized PARylated-PARP-1 but not MARylated PARP-3, or the precursors of ADPR (FIG. 1C). Accordingly, the utility of these reagents was further tested in in vitro PARP-1 PARylation reactions with increasing concentrations of NAD+(FIGS. 6F and 6G) and increasing time of reaction (FIG. 1D and FIG. 1E). Similarly, degradation of PAR chains by ARH3 was detected in a time (FIG. 1F and FIG. 1G) and dose (FIG. 6H) dependent way. Further, in vitro reactions were performed by incubating the recombinant PAR-T sensors and lysates from Hela cells treated with H2O2 to induce DNA damage and activate PARP-1. H2O2 and PARG inhibitor treatments resulted in an increase in fluorescence that was lowered in PARP inhibitor treated cells. Further, a pronounced increase in fluorescence was observed when the lysates from cells treated with both H2O2 and PARG inhibitor were used (FIG. 6I and FIG. 6J). Together, this data suggests that the PAR-T sensors can specifically recognize PARylation, and they can be used in time course experiments in vitro.

### Example 2: Using Dimerization-Dependent GFP-Based Reagents to Detect PARylation in Live Cells

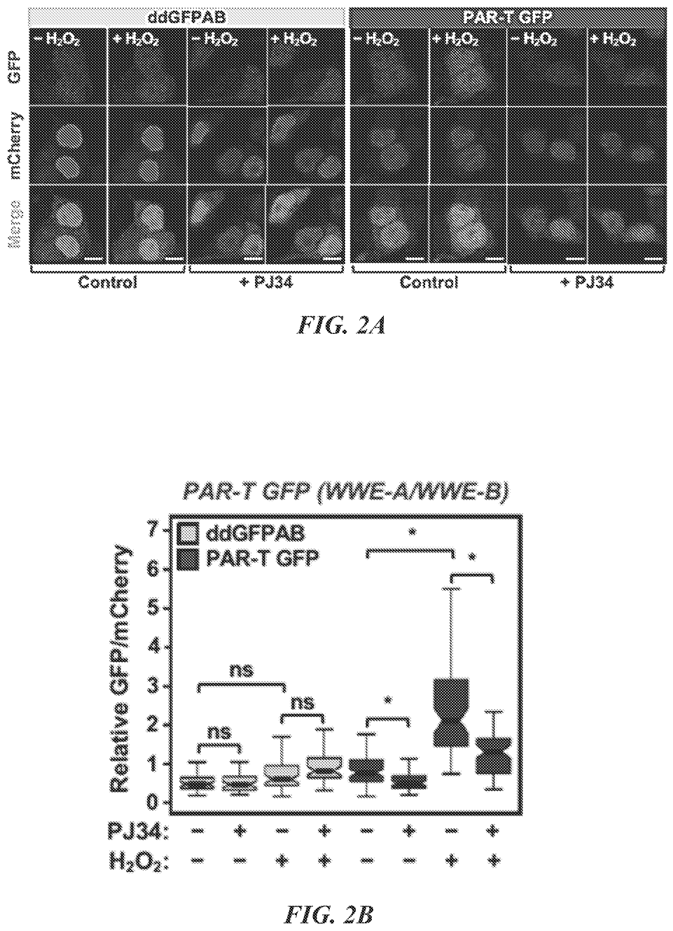

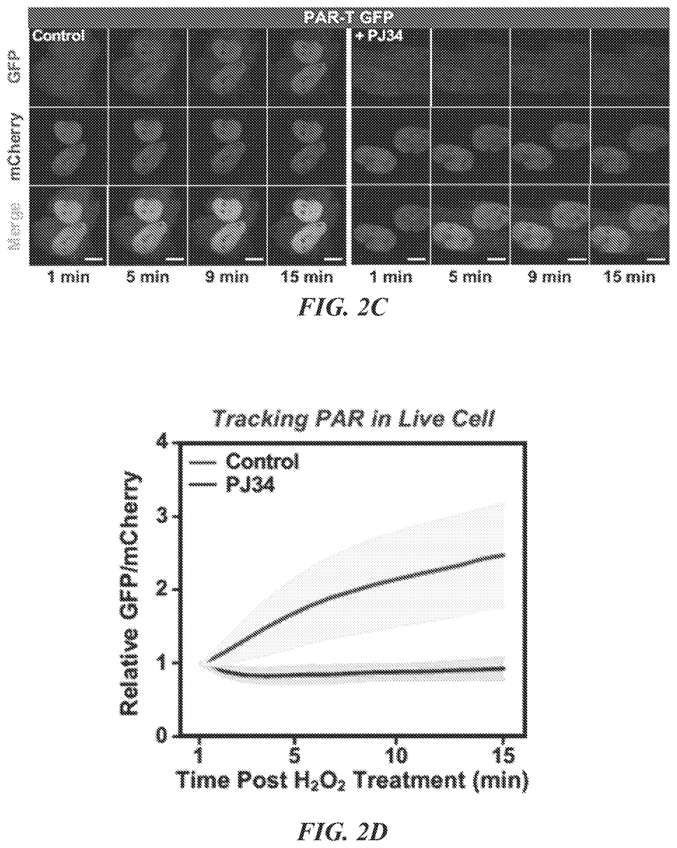

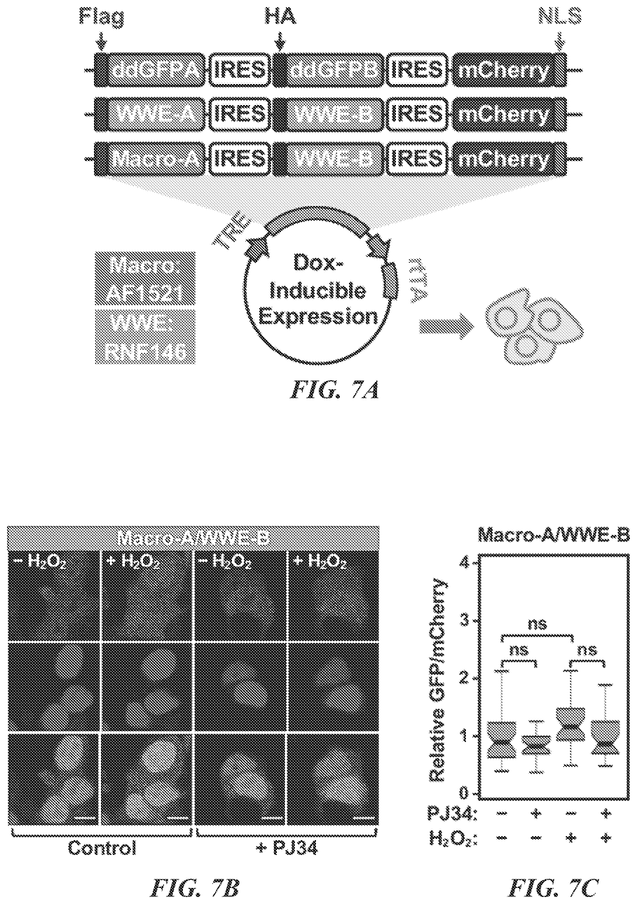

Having confirmed the specificity of the PAR-T sensors, their utility in live cell imaging was then tested. PAR-T sensors were expressed in Hela cells in a doxycycline-dependent manner and live cell imaging performed after subjecting the cells to H₂O₂-mediated PARP-1 activation (FIG. 7A). This PAR-T construct also expresses mCherry with nuclear localization signal (NLS) to illuminate the nuclei and act as a control for variability in expression of the constructs (FIG. 7A). When compared to ddGFP alone, ddGFP-conjugated to WWE detected PARP-1 activation in live cell imaging (FIG. 2A and FIG. 2B). Interestingly, even though the WWE-macrodomain combination sensor was able to detect PARylation in vitro, this sensor combination failed to recognize PARylation in cells (FIG. 7B and FIG. 7C). Hence, the WWE based PAR-T sensors were used for subsequent Examples. Using the WWE-based ddGFP PAR-T construct, accumulation of PAR after H₂O₂-treatment was detected in real time. Treatment with PARP inhibitor blocked this accumulation (FIG. 2C and FIG. 2D).

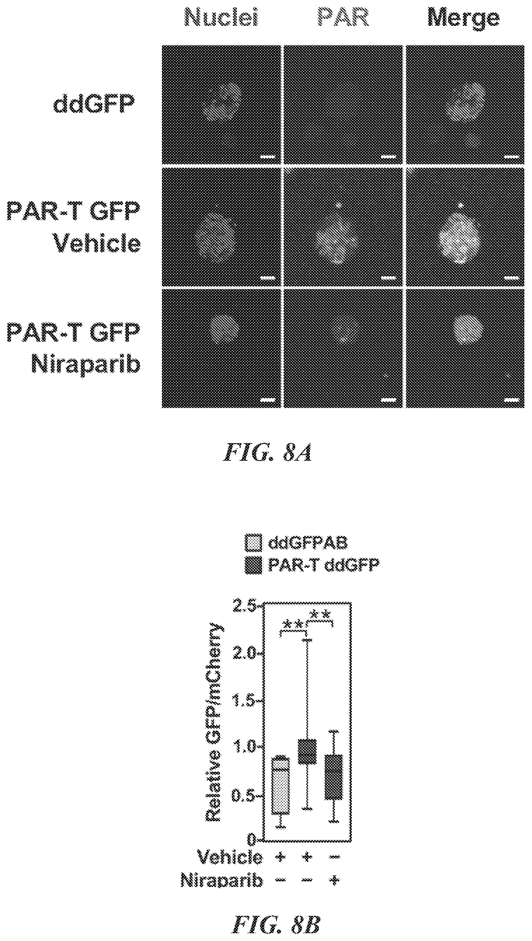

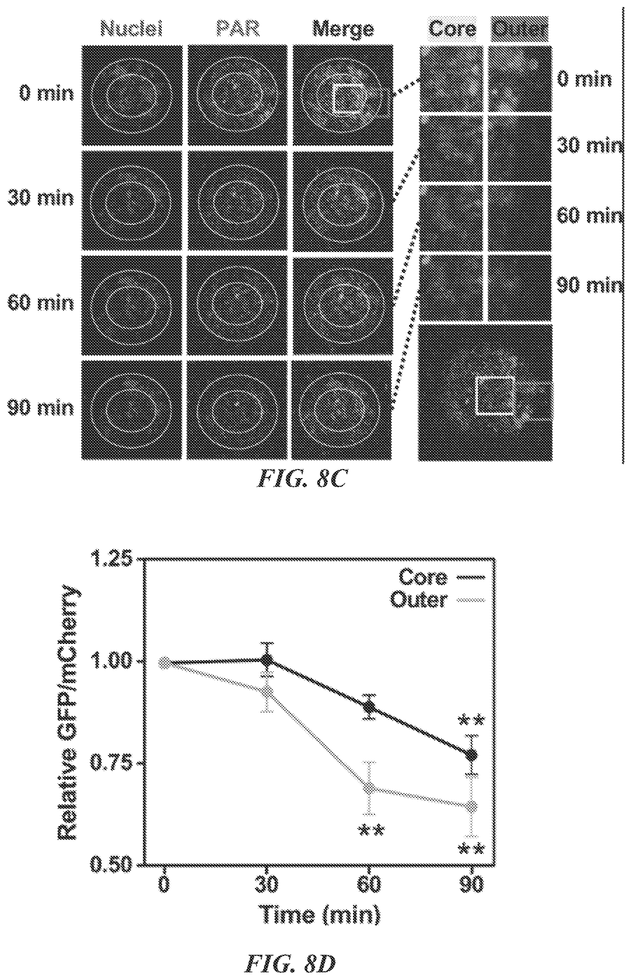

Cancers are heterogenous tissues with spatial variation in nutrient availability, and stress. Since PARylation is enhanced by stressors such as DNA damage, another experiment tested whether there was any spatial variation in PARylation levels. Live cell imaging was performed in 3D cancer spheroids using MCF7 cells expressing the WWE based PAR-T sensors. A spatial distribution of PARylation was observed throughout the spheroid, that was inhibited by the PARP inhibitor, olaparib (FIG. 8A). A heterogeneous distribution of PAR throughout the spheroid was observed, which was inhibited by the PARP inhibitor, Niraparib (FIG. 8A-FIG. 8B). A time course of Niraparib treatment in 3D cancer spheroids was performed to visualize spatio-temporal changes in PAR levels over time. The results indicate that the PAR levels in cells at the core of the spheroids are relatively resistant to Niraparib treatment, since the PAR levels in these cells decrease at a lower rate compared to the PAR levels in the cells in the outer layer of the spheroid (FIG. 8C and FIG. 8D). These data provide evidence that the ddGFP based PAR-T sensors can be used for live cell imaging to evaluate the spatial and temporal changes of PARylation in cancer cells.

### Example 3: Developing a Highly Sensitive Split-Luciferase PAR-T Detection Reagent

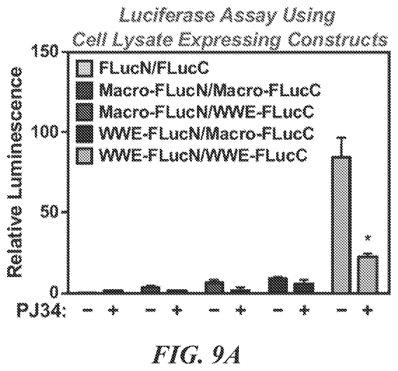

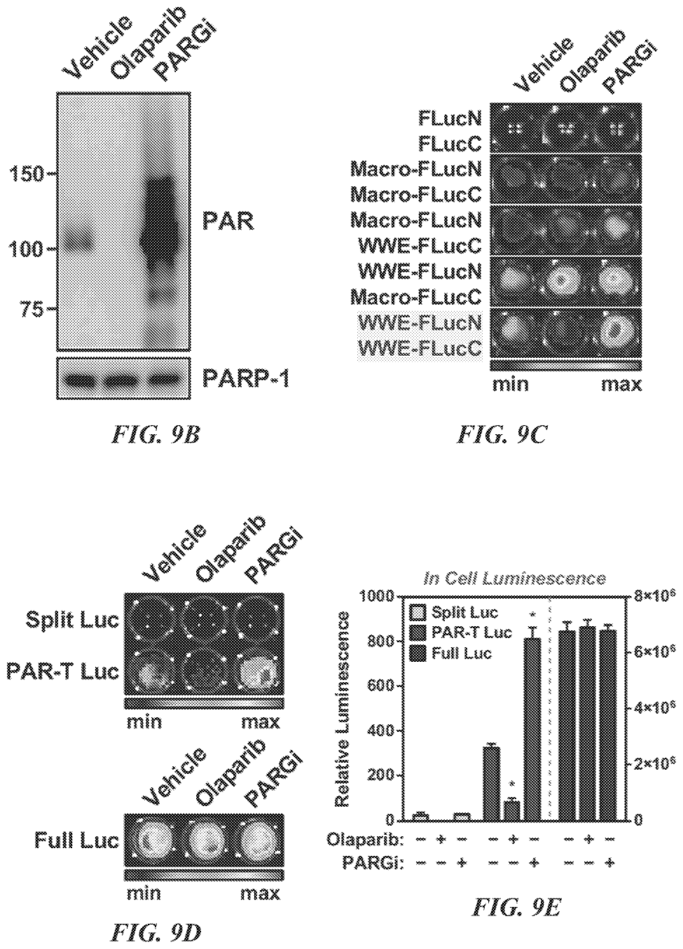

The previous examples showed that WWE-domain based PAR-T sensors can detect PARylation specifically. In this example, a set of highly sensitive PAR-T reagents were developed to detect PARylation in vivo. In a first set of experiments, split firefly luciferase reagents were generated using various combinations of the ARBDs. WWE domains performed consistently better in identifying an increase in PARylation with PARG inhibitor and decrease in PARylation with a PARP inhibitor treatment using both the cell lysates (FIG. 9A) and in cells (FIG. 9B and FIG. 9D). Luminescence from an unsplit Firefly luciferase remain unaltered with these treatments (FIG. 9E and FIG. 9F).

However, the bulkiness of firefly luciferase may interfere with the function of the domains fused to them in complementation assays. Accordingly, a sensor was constructed with a newer luciferase protein, Nano Luciferase or NanoLuc, that is more stable and brighter to see whether it could have a better performance in vivo compared to firefly luciferase. Further, since the C-terminal region of the split nano luciferase was unstable, a WWE-based split Nano Luciferase construct was prepared with the C-terminal domain fused to a fluorophore, LSSmOrange.

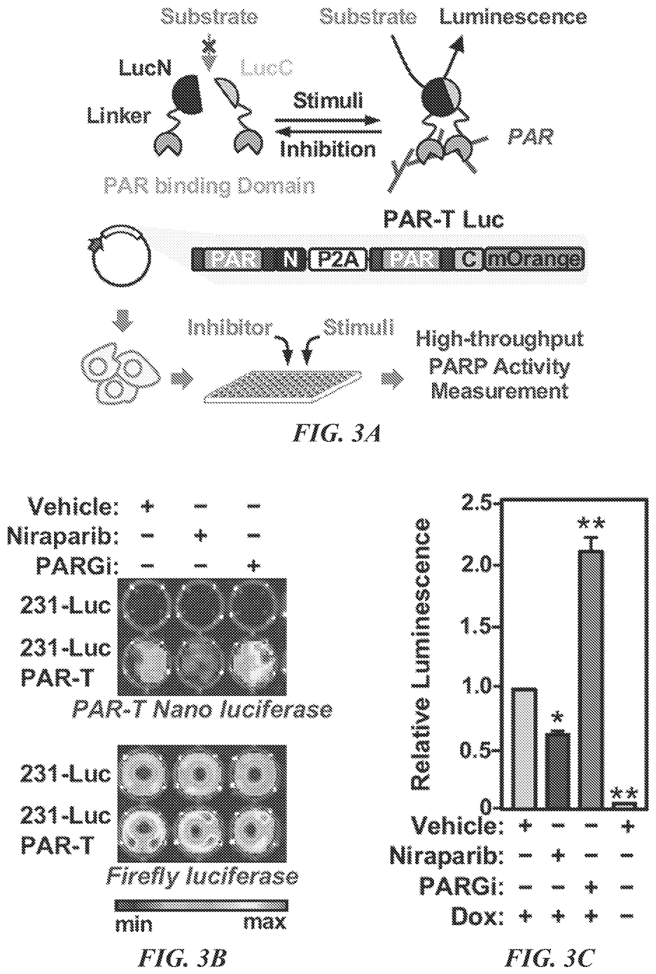

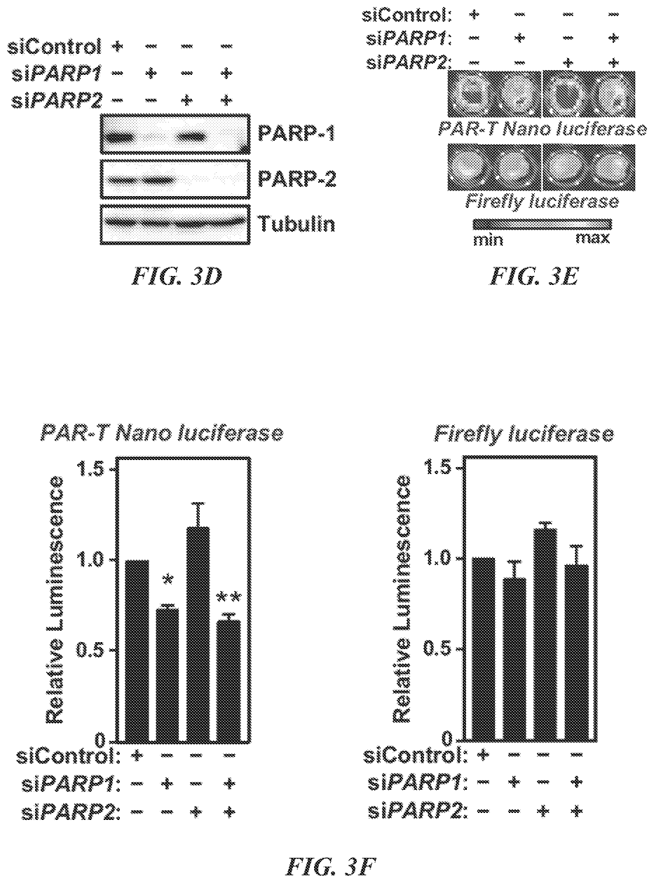

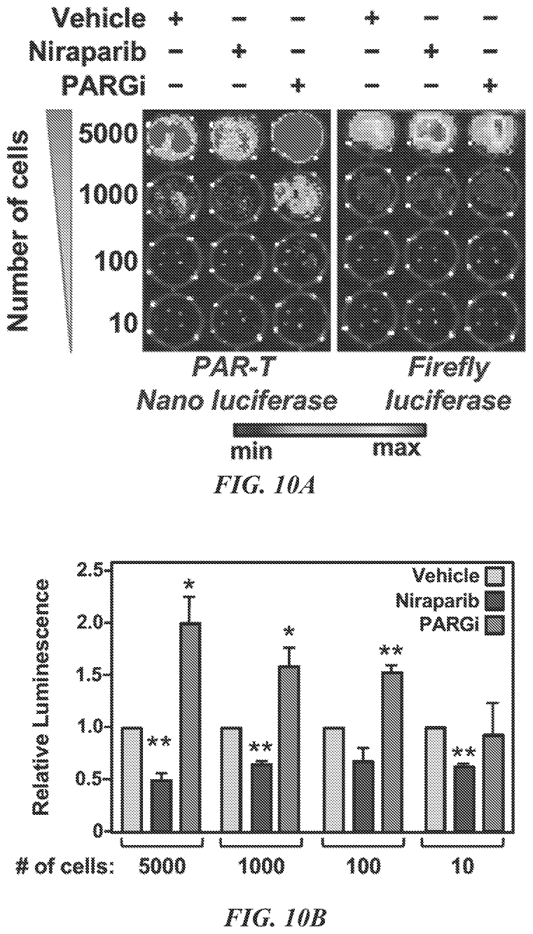

In order to have a better quantitative analysis of this sensor, the luminescence PAR-Tracker was expressed in a doxycycline-dependent manner in human breast cancer cells that have stable expression of firefly luciferase (MDA-MB-231-Luc cells) (FIG. 3A). This way, changes in cell viability or tumor size could be normalized across experiments. First, it was tested if there is cross reactivity of the two luciferases to the substrates. Specific detection of firefly luciferase with D-Luciferin and Nano luciferase with furimazine was observed with no cross-reactivity (FIG. 3B and FIG. 3C). PARP-1 depletion reduced the luminescence from PAR-T nano Luciferase with little effect on luminescence of firefly luciferase (FIG. 3D, FIG. 3E and FIG. 3F). Intriguingly, knockdown of PARP-2 has no effect on luminescence from PAR-T nano Luciferase. Nevertheless, the luminescent PAR-T sensor is extremely sensitive and can be used to detect PARylation in just 1000 cells, with a dynamic range of approximately two-fold (FIG. 10A and FIG. 10B).

### Example 4: Detection of Radiation-Induced PARP-1 Activation in Breast Cancer Cells

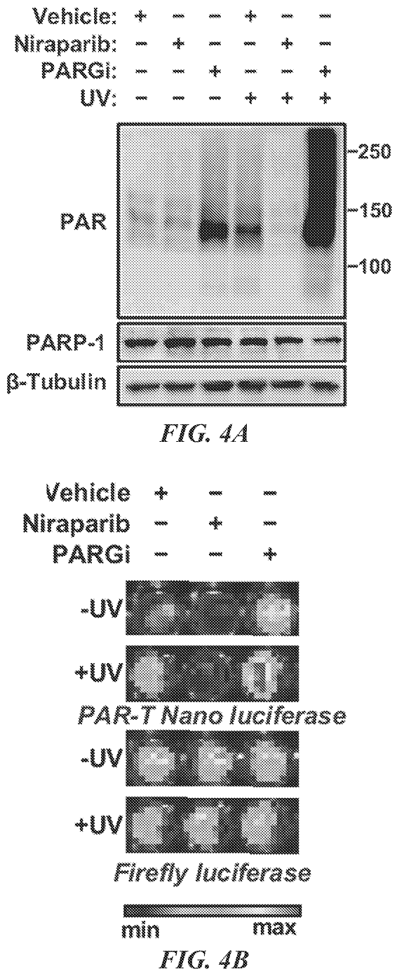

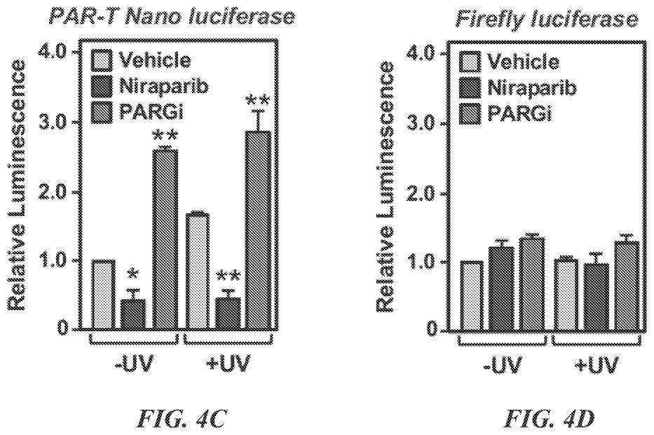

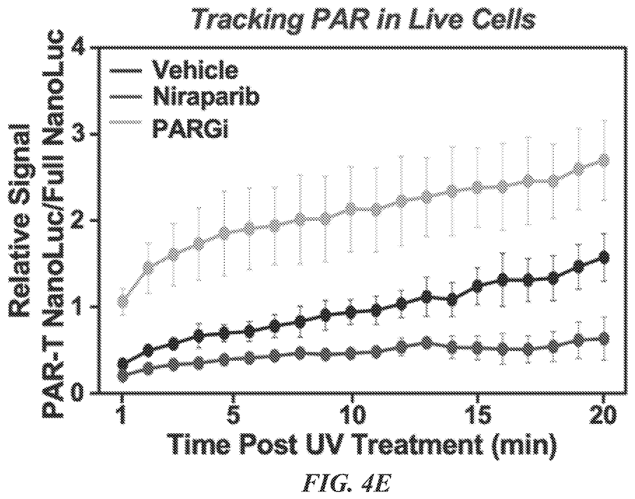

DNA damaging agents such as UV irradiation and y irradiation activate PARP-1 and cause PARylation of itself and other DNA damage repair proteins that are recruited to the damage sites. Since the PAR-T sensor was able to detect H₂O₂-induced PARP-1 activation (FIG. 2), it was then tested to see if it could detect radiation-induced PARP-1 activation. MDA-MB-231-Luc cells were subjected to doxycycline-induced expression of PAR-T, and then treated UV radiation. Indeed, UV radiation induced PARP-1 activation, that was further enhanced by inhibition of PARG, but treating with the PARP inhibitor inhibited this UV-induced PARP activation (FIG. 4A). UV radiation of PARG inhibitor treated cells enhanced PAR-T luminescence, but PARP inhibitor treatment reduced the PAR-T luminescence (FIG. 4B and FIG. 4C). None of these treatments had a significant effect on the luminescence from firefly (FIG. 4B and FIG. 4D). In a similar manner, a time course of UV-mediated PARP-1 activation using live cell luminescence assay with the PAR-T NanoLuc sensor was performed. The MDA-MB-231 cells were subjected to Dox-induced expression of PAR-T NanoLuc or intact Nano luciferase and then exposed the cells to UV radiation. Consistent with the previous experiment, observed was a time-dependent increase in PAR-T NanoLuc signal in vehicle-treated cells, but not in Niraparib-treated cells. Interestingly, UV-mediated increases in PARP-1 activation were more spontaneous in PARG inhibitor-treated cells (FIG. 4E). The PAR levels under basal (-UV) conditions were low, resulting in only a 50% decrease in PAR-T NanoLuc signal with Niraparib treatment (FIG. 4A-FIG. 4C). The decrease in PAR-T NanoLuc signal was greater when UV-treated cells were pre-treated with Niraparib, which is consistent with the results from Western blot analysis (FIG. 4A).

### Example 5: Comparison of Assay Performance Using the PAR-T Sensor and Conventional PAR Detection Reagents

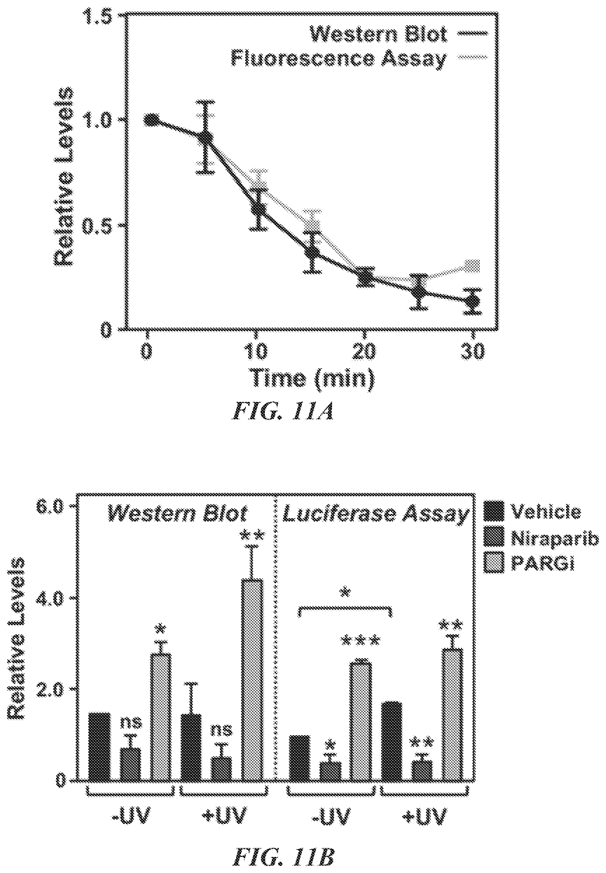

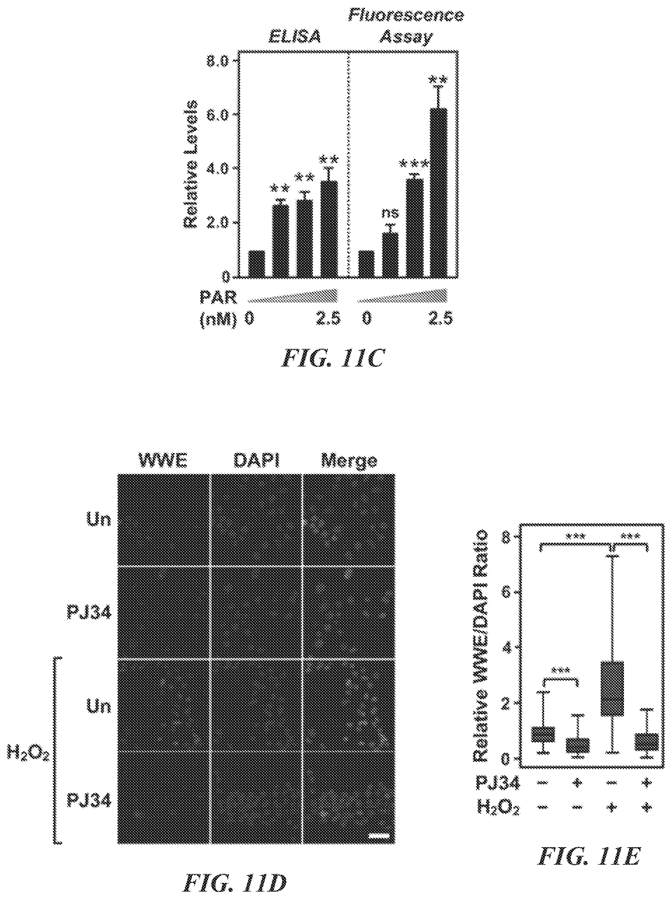

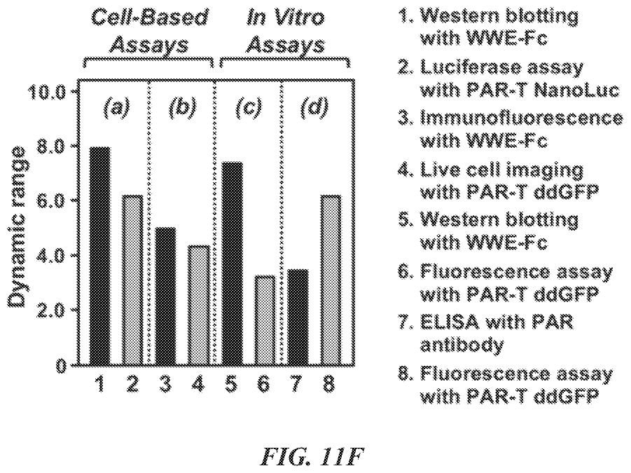

Next, a set of assays was performed to compare the performance of the PAR-T sensors to conventional PAR detection reagents (WWE-Fc and PAR antibody) in a variety of assays. The following assays were compared (1) Western blotting with WWE-Fc versus fluorescence assay with PAR-T ddGFP, which were performed in conjunction with ARH3-mediated degradation of PAR in vitro (FIG. 11A); (2) Western blotting with WWE-Fc versus live-cell luciferase assay using PAR-T NanoLuc, which were performed in conjunction with UV-induced DNA damage in MDA-MB-231 cells (FIG. 11B); (3) enzyme-linked immunosorbent assay (ELISA) with PAR antibody versus fluorescence assay with PAR-T ddGFP, which were performed using immobilized PAR (FIG. 11C); and (4) immunofluorescence with WWE-Fc versus live-cell imaging using PAR-T ddGFP, which were performed using H2O2-mediated PARP-1 activation in 293T cells (FIG. 11D and FIG. 11E).

The dynamic ranges of the PAR-T sensors were compared with the other reagents (WWE-Fc and PAR antibody) in various PAR detection assays, such as Western blotting and ELISA (FIG. 11F). Western blotting with WWE-Fc had the highest dynamic range for detection of PAR (eightfold), but the dynamic range of live-cell luciferase assay with PAR-T NanoLuc was comparable (sixfold) (FIG. 11F). While PAR-T ddGFP in a modified fluorescence assay had a larger dynamic range than PAR antibody in an ELISA (6-fold vs. 3.5-fold) when the assays were performed using immobilized PAR, it had a lower dynamic range when used for live-cell imaging (4.4-fold). This can be explained, in part, by the higher autofluorescence of cells, which can diminish the dynamic range of the PAR-T ddGFP sensors. Thus, this sensor may require further optimization to increase the signal-noise ratio. Nevertheless, the performance of PAR-T ddGFP in live cells is comparable to that of an immunofluorescence assay with WWE-Fc (4.4-fold vs. 5-fold).

### Example 6: Detection of PAR Production from PARP-1 Activation Under Physiological Conditions

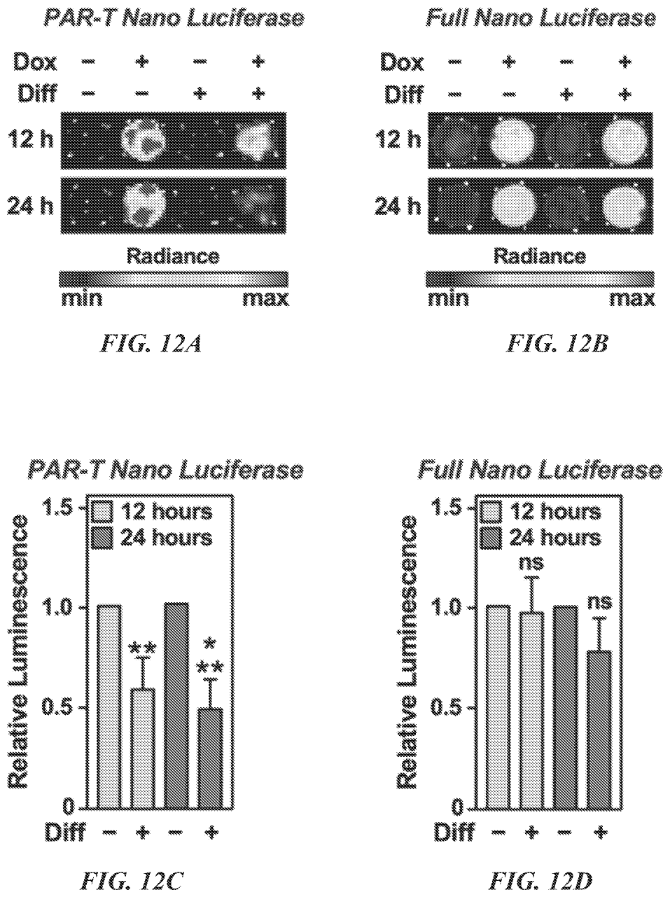

PARP-1 catalytic activity decreases during the initial differentiation of preadipocytes. Thus, adipogenesis is a unique biological process to study the dynamics of PAR accumulation from changes in PARP-1 activity under physiological conditions. To this end, the PAR-T NanoLuc sensor was used to investigate changes in PARP-1 activity during early adipogenesis of murine preadipocytes (i.e., 3T3-L1 cells). A decrease in the signal from PAR-T NanoLuc was observed by 12 hr of differentiation and a greater reduction in PAR-T NanoLuc signal noted by 24 hr of differentiation (FIG. 12A-FIG. 12D), consistent with previous observations that PARP-1 activation decreases precipitously during adipogenesis. These results further highlight the high sensitivity of PAR-T NanoLuc sensor, which can be used to study physiological changes in PAR levels during biological processes, such as adipogenesis.

### Example 7: Detection of PAR Production from PARP-1 Activation In Vivo

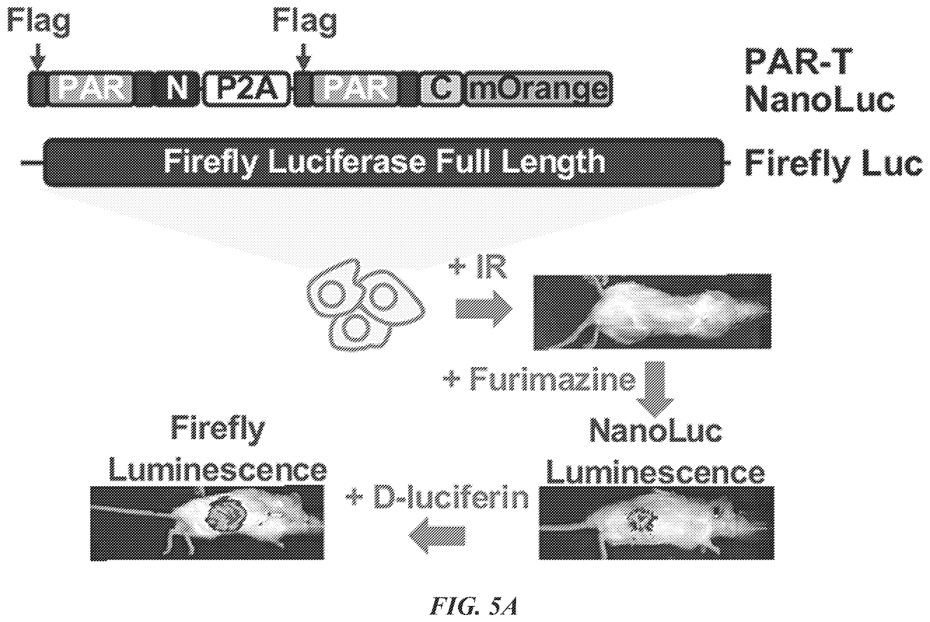

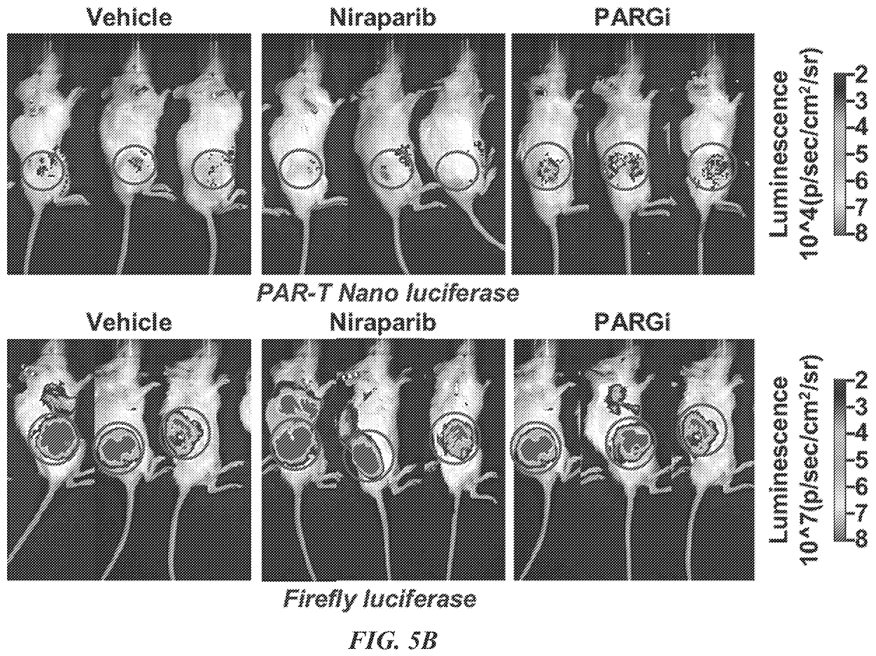

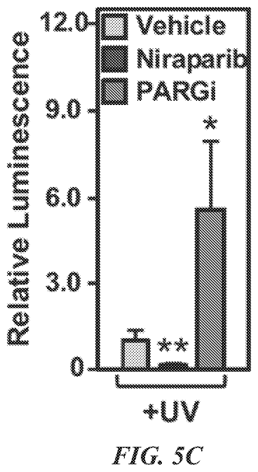

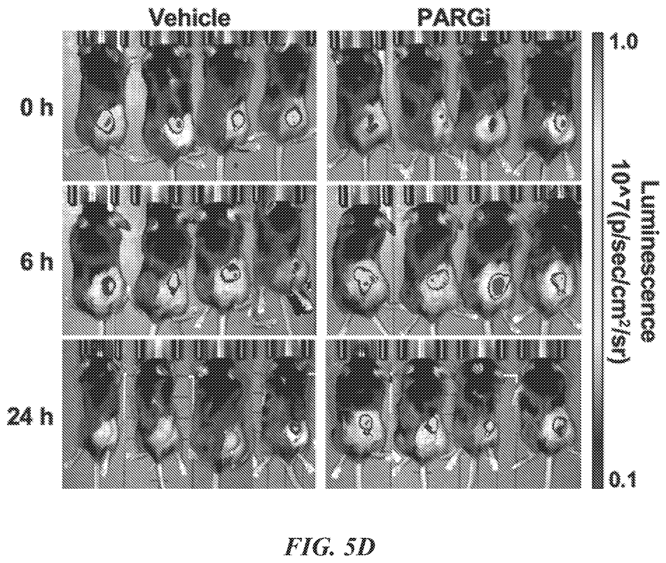

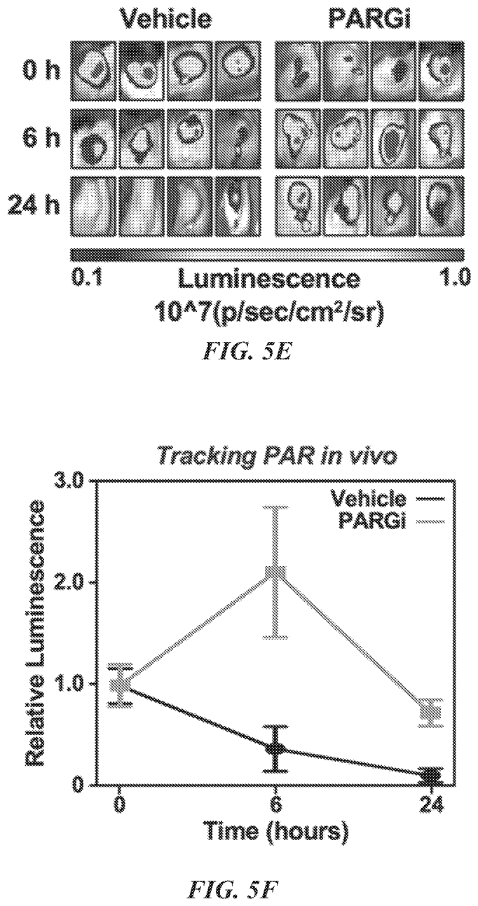

In the next experiment, the utility of luminescent PAR-T sensor to measure the levels of PARylation in vivo was tested. Xenograft tumors were established using the MDA-MB-231-luc cells the expression of PAR-T was induced by doxycycline (FIG. 5A). Similar to the in vitro experiments, an increase in PARylation using PAR-T sensors was detected when the mice were treated with both y irradiation and PARG inhibitors, but the luminescence was decreased when the mice were treated with PARP inhibitor (FIGS. 5B and 5C). The luminescence from PAR-T was normalized to the firefly luminescence to confidently measure the differences in PARylation, while accounting for the variability in tumor sizes.

PAR accumulation in breast cancer cells injected into C57/BL6 mice without establishing xenograft tumors over a 24-hr time course post injection, with or without PARG inhibitor treatment (FIG. 5D, FIG. 5E and FIG. 5F) was measured. PAR accumulation was readily detected in the breast cancer cells injected into the mice in the absence of treatment. Upon treatment with PARG inhibitor, the luminescence from PAR-T NanoLuc increased significantly by 6 hr and then diminished by 24 hr. These results demonstrate that the PAR-T NanoLuc sensor has sufficient sensitivity to detect dynamic changes in PAR production in tissues of living animals in vivo.

### Discussion of Examples 1 to 7

Naturally occurring ADPR binding domains are invaluable for developing novel ADPR detection reagents. In Examples 1 to 4, a set of exemplary ADPR detection reagents were developed that are useful tools for use in in vitro assays, in live cells, and in animals. For example, a fluorescence-based PAR-Tracker was shown to be useful for in vitro assays and live cell imaging, and to track PARylation levels at a single cell level (FIG. 2). In contrast to related sensors that relied on PBZ binding domains, the experiments described herein show that a WWE domain (e.g., from RNF146) and a macrodomain (e.g., from AF1521) worked better as PAR binding domains for detection of PARP-1 activation, compared to PBZ domains from APLF (FIG. 6E).

Fluorescence sensors are not optimal for use in vivo, due to high auto-fluorescence of tissues. Hence Example 3 describes an exemplary luminescence-based sensor to increase the sensitivity of PAR detection in vivo. Several aspects of the sensor were optimized to achieve highest sensitivity: (1) use of Nano luciferase, the smallest and brightest luciferase available, (2) addition of mOrange to stabilize the C-terminal fragment of Nano Luciferase and overcome tissue penetration issues, (3) using the Nano-Glo live cell substrate in in vitro cell-based assays to be able to perform these assays in live cells, (4) use doxycycline-inducible constructs to avoid any effects of expression of these constructs on cell viability, (5) developing a dual luciferase assay to quantify PARylation levels more accurately. Indeed, the luminescent PAR-T sensor can detect PARylation levels in as few as 1000 cells with good dynamic range of detection (FIG. 10A and FIG. 10B). This is the first report to describe a tool capable of detecting PARylation levels in living animals.

Recently, major efforts are directed at identification of better ADPR detection tools, to improve drug discovery. Similarly, recent studies have developed BRET sensors to study target engagement of PARP enzymes. Use of this Nano Luciferase based BRET sensors has improved the sensitivity of these assays. Similarly, using the Nano luciferase PAR-T sensor has improved the sensitivity to detect PARylation, thus enhancing the capability of this assay for use in high throughput screens, or to measure PARylation levels in limited amount of samples such as tissues from mice, and clinical patient samples. Taken together, these findings make the PAR-Trackers an immense improvement to detection reagents with broader in vitro, in cellulo, and in vivo applications

Having described several embodiments, it will be recognized by those skilled in the art that various modifications, alternative constructions, and equivalents may be used without departing from the spirit of the invention. Additionally, a number of well-known processes and elements have not been described in order to avoid unnecessarily obscuring the present invention. Accordingly, the above description should not be taken as limiting the scope of the invention.

Those skilled in the art will appreciate that the presently disclosed embodiments teach by way of example and not by limitation. Therefore, the matter contained in the above description or shown in the accompanying drawings should be interpreted as illustrative and not in a limiting sense. The following claims are intended to cover all generic and specific features described herein, as well as all statements of the scope of the present method and system, which, as a matter of language, might be said to fall therebetween.

1. A split reporter system for detecting poly-ADP ribose polymerase (PARP) activity comprising:
(a) a first fusion protein comprising a first fragment of a reporter protein functionally linked to a first poly-ADP ribose binding moiety; and
(b) a second fusion protein comprising a second fragment of the reporter protein functionally linked to a second poly-ADP ribose binding moiety;
wherein the first and second fragments of the reporter protein are each non-functional and capable of recombining, optionally in the presence of a substrate, to form a functional reporter protein capable of producing a detectable signal.

2. The split reporter system of claim 1, wherein the reporter protein comprises a fluorescent or a luminescent protein.

3. (canceled)

4. The split reporter system of claim 2, wherein the reporter protein comprises a luciferase.

5-9. (canceled)

10. A split reporter system for detecting poly-ADP ribose polymerase (PARP) activity comprising:
(a) a first fusion protein comprising a first monomer of a dimerization-dependent reporter system functionally linked to a first poly-ADP ribose binding moiety; and
(b) a second fusion protein comprising a second monomer of the dimerization dependent reporter system functionally linked to a second poly-ADP ribose binding moiety;
wherein the first and second monomers of the dimerization dependent reporter system are capable of combining to form a heterodimer of the dimerization-dependent reporter system, the heterodimer capable of emitting a detectable light signal.

11. The split reporter system of claim 10, wherein the dimerization dependent reporter system comprises a dimerization-dependent GFP, a dimerization-dependent YFP, or a dimerization dependent RFP.

12. (canceled)

13. The split reporter system of claim 10, wherein the first and/or second monomers are operably linked to a second fluorescent protein excited by light emitted from the heterodimer when the heterodimer is excited by electromagnetic radiation.

14. The split reporter system of claim 13, wherein the second fluorescent protein comprises mOrange, cpVenus or GFP.

15. The split reporter system of claim 10, wherein at least one of the first or second poly-ADP ribose binding moieties comprise a macro domain.

16. The split reporter system of claim 15, wherein the macro domain comprises a macro domain derived from an ADP ribose glycohydrolase AF1521.

17. The split reporter system of claim 16, wherein the macro domain comprises an amino acid sequence having at least 80% sequence identity to SEQ ID NO: 1.

18. The split reporter system of claim 10, wherein at least one of the first and second poly-ADP ribose binding moieties comprises a WWE domain.

19. The split reporter system of claim 18, wherein the WWE domain comprises a WWE domain derived from an RNF146 E3 ligase.

20. The split reporter system of claim 19, wherein the WWE domain has an amino acid sequence having at least 80% sequence identity to SEQ ID NO: 2.

21. The split reporter system of claim 10, wherein the first and second poly ADP ribose binding moieties lack a PBZ domain having an amino acid sequence comprising SEQ ID NO: 3.

22. A nucleic acid construct comprising a nucleic acid sequence that encodes for the first and/or second fusion protein of claim 10.

23. An expression vector comprising at least one nucleic acid construct of claim 22.

24. A host cell comprising one or more expression vectors of claim 23.

25. A method of detecting poly-ADP ribose polymerase (PARP) activity in a cell or tissue suspected of having PARP activity, the method comprising:
(a) introducing the first and second fusion proteins of claim 10 into the cell or tissue;
(b) maintaining the cell or tissue for a time and under conditions sufficient for the first and second fusion proteins to bind to one or more poly-ADP ribose (PAR) chains and combine to produce a signal; and
(c) detecting the signal, wherein the signal is proportional to the PARP activity in the system.

26. A method of assessing the effectiveness of a potential therapeutic comprising (a) introducing the first and second fusion proteins of claim 10 into a cell, tissue or animal, (b) applying the potential therapeutic to the cell or tissue, (c) maintaining the cell or tissue for a time and under conditions sufficient for the first and second fusion proteins to bind to one or more poly-ADP ribose (PAR) chains and combine to produce a signal; and (d) detecting the signal, wherein the signal is indicative of the efficacy of the potential therapeutic.

27-36. (canceled)

37. A kit comprising one or more of the first or second fusion proteins of claim 10 and at least one container.
